SEQUENCE LISTING (2 sequences)

SEQ ID NO: 1 - protein, 223 aa, Artificial sequence
  Synthetic polypeptide
EVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 2 - protein, 214 aa, Artificial sequence
  Synthetic polypeptide
EIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

(19) United States
(12) Patent Application Publication
(10) Pub. No.: US 2010/0150864 A1
(43) Pub. Date: Jun. 17, 2010

(54) ANTIBODIES THAT BIND TO IL-18 AND METHODS OF PURIFYING THE SAME

(75) Inventors: Robert K. Hickman, Worcester, MA (US); Qing Huang, Worcester, MA (US); Johanna Gervais, West Townsed, MA (US)

Correspondence Address: BAKER BOTTS L.L.P., 30 ROCKEFELLER PLAZA, 44th Floor, NEW YORK, NY, 10112-4498, US

(73) Assignee: Abbott Laboratories, Inc., Abbott Park, IL (US)

(21) Appl. No.: 12/582,469

(22) Filed: Oct. 20, 2009

Related U.S. Application Data
(60) Provisional application No. 61/196,751, filed on Oct. 20, 2008.

(51) Int. Cl. C07K 16/24; A61K 39/395; A61K 38/20

(52) U.S. Cl. 424/85.2; 530/387.1; 530/387.3; 424/158.1; 530/391.3

(57) ABSTRACT

Anti-IL-18 antibodies are disclosed herein, including antigen-binding portions thereof. One or more methods for isolating and purifying anti-IL-18 antibodies from a sample matrix is presented. These isolated anti-IL-18 antibodies can be used in a clinical setting as well as in research and development. Pharmaceutical compositions comprising isolated anti-IL-18 antibodies are also described.

### CROSS REFERENCE TO RELATED APPLICATIONS

This application claims the benefit of U.S. Provisional Application Ser. No. 61/196,751, filed Oct. 20, 2008, which is hereby incorporated by reference in its entirety.

### BACKGROUND OF THE INVENTION

Human interleukin-18 is an identified cytokine that is synthesized as a biologically inactive 193 amino acid precursor protein. Cleavage of the precursor protein, e.g., by caspase-1 or caspase-4, liberates a 156 amino acid mature protein that exhibits biological activities that include the co-stimulation of T cell proliferation, the enhancement of NK cell cytotoxicity, the induction of IFN-γ production by T cells and NK cells, and the potentiation of T helper type 1 (Th1) differentiation. In addition, IL-18 is an efficacious inducer of human monocyte proinflammatory mediators, including IL-8, tumor necrosis factor-α (TNF-α), and prostaglandin E₂ (PGE₂).

IL-18 plays a potential role in immunoregulation or in inflammation by augmenting the functional activity of Fas ligand on Th1 cells. IL-18 is also expressed in the adrenal cortex and therefore might be a secreted neuro-immunomodulator, playing an important role in orchestrating the immune system following a stressful experience.

Th1 cells, which produce pro-inflammatory cytokines such as IFN-γ, IL-2 and TNF-β have been implicated in mediating many autoimmune diseases, including multiple sclerosis (MS), rheumatoid arthritis (RA), type 1 or insulin dependent diabetes (IDDM), inflammatory bowel disease (IBD), and psoriasis. Thus, antagonism of a TH1-promoting cytokine such as IL-18 would be expected to inhibit disease development. Il-18 specific mAbs could be used as an antagonist.

In vivo, IL-18 is formed by cleavage of pro-IL-18, and its endogenous activity appears to account for IFN-γ production in P. acnes and LPS-mediated lethality. Blocking the biological activity of IL-18 in human disease is a therapeutic strategy in many diseases. This can be accomplished using soluble receptors or blocking antibodies to a cell-bound IL-18 receptor.

Cytokine binding proteins (soluble cytokine receptors) correspond to the extracellular ligand binding domains of their respective cell surface cytokine receptors. They are derived either by alternative splicing of a pre-mRNA, common to the cell surface receptor, or by proteolytic cleavage of the cell surface receptor. Such soluble receptors have been described in the past, including, among others, the soluble receptors of IL-6 and IFN-γ. One cytokine-binding protein, named osteoprotegerin (OPG, also known as osteoclast inhibitory factor—OCIF), a member of the TNFR/Fas family, appears to be the first example of a soluble receptor that exists only as a secreted protein.

It has been suggested that IL-18 is involved in the progression of pathogenicity in chronic inflammatory diseases, including endotoxin shock, hepatitis, and autoimmune diabetes. A possible role of IL-18 in the development of liver injury was postulated based on experiments showing an elevated level of IL-18 in lipopolysaccharide-induced acute liver injury in a mouse model. However, the mechanism of the multi-functional factor IL-18 in the development of liver injury has not been elucidated so far.

Recent studies indicate that IL-18 plays a pro-inflammatory role in joint metabolism. Investigators showed that IL-18 is produced by articular chondrocytes and induces pro-inflammatory and catabolic responses. IL-18 mRNA was induced by IL-1β in chondrocytes. Chondrocytes produced the IL-18 precursor and, in response to IL-1 stimulation, secreted the mature form of IL-18. Studies on IL-18 effects on chondrocytes further showed that it inhibits TGF-β-induced proliferation and enhances nitric oxide production. IL-18 stimulated the expression of several genes in normal human articular chondrocytes including inducible nitric oxide synthase, inducible cyclooxygenase, IL-6, and stromelysin. Gene expression was associated with the synthesis of the corresponding proteins. Treatment of normal human articular cartilage with IL-18 increased the release of glycosaminoglycans. These finding identified IL-18 as a cytokine that regulates chondrocyte responses and contributes to cartilage degradation.

It has been suggested that IL-18 plays a pro-inflammatory role in rheumatoid arthritis. IL-18 levels have been shown to be markedly elevated in the synovial fluid of rheumatoid arthritis patients. Investigators have detected the IL-18 mRNA and protein within rheumatoid arthritis synovial tissues in significantly higher levels than in osteoarthritis controls. It was also shown that a combination of IL-12 or IL-15 with IL-18 induced the IFN-γ production by synovial tissues in vitro. Furthermore, IL-18 administration of collagen/incomplete Freund's adjuvant-immunized mice facilitated the development of an erosive, inflammatory arthritis, suggesting that IL-18 may be proinflammatory in vivo.

The role of IL-18 in the development of other autoimmune diseases has been demonstrated. Accordingly, it has been demonstrated that IL-18 expression is significantly increased in the pancreas and spleen of the nonobese diabetic (NOD) mouse immediately prior to the onset of disease. Furthermore, it has been demonstrated that IL-18 administration increases the clinical severity of murine experimental allergic encephalomyelitis (EAE), a Th1-mediated autoimmune disease that is a model for multiple sclerosis. In addition, it has been shown that neutralizing anti-rat IL-18 antiserum prevents the development of EAE in female Lewis rats. Accordingly, IL-18 is a desirable target for the development of a novel therapeutic for autoimmunity.

IL-18 is a pleiotropic interleukin having both inflammatory enhancing and attenuating functions. On the one hand, it enhances production of the pro-inflammatory cytokines like TNF-α, therefore promoting inflammation. On the other hand, it induces the production of NO, an inhibitor of caspase-1, thus blocking the maturation of IL-1β and IL-18, and possibly attenuating inflammation. This ambiguous role of IL-18 raised questions as to the efficacy of IL-18 inhibitors in treating inflammatory diseases. Furthermore, because of the interaction of a wide variety of different cytokines and chemokines in the regulation of inflammation, it could not have been expected that a beneficial effect would be obtained by blocking only one of the players in such a complicated scenario.

Notwithstanding the foregoing, neutralizing IL-18 antibodies are considered useful in relieving autoimmune diseases and related symptoms. Hence there is a need in the art for a high affinity IL-18 antibody, such as a neutralizing monoclonal antibody to human interleukin 18. Furthermore, it is important that a therapeutic regime comprising antibodies against IL-18 be of high purity. The present invention addresses this need without the use of a Protein A column or an equivalent Protein A-based purification step.

### SUMMARY OF THE INVENTION

In certain embodiments, the present invention is directed to purified, isolated antibodies and antibody fragments that bind to IL-18 as well as pharmaceutical compositions comprising such antibodies and fragments. In certain embodiments, the invention pertains to isolated antibodies, or antigen-binding portions thereof, that bind to human IL-18. The isolated anti-IL-18 antibodies of the present invention can be used in a clinical setting as well as in research and development. In certain embodiments, the present invention is directed to the anti-IL-18 antibody comprising the heavy and light chain sequences identified in SEQ ID NOs. 1 and 2.

Certain embodiments of the invention are directed toward methods of purifying anti-IL-18 antibodies, or antigen-binding portions thereof, from a sample matrix to render them substantially free of host cell proteins (“HCPs”). In certain aspects, the sample matrix (or simply “sample”) comprises a cell line employed to produce anti-IL-18 antibodies of the present invention. In particular aspects, the sample comprises a cell line used to produce human anti-IL-18 antibodies.

In certain embodiments of the present invention a sample matrix comprising the putative anti-IL-18 antibody, or antigen-binding portion thereof, is subjected to a pH adjustment. In certain aspects, the pH is adjusted to about 3.5. The low pH, among other things, promotes the reduction and/or inactivation of pH-sensitive viruses that may be contaminating the sample. After a suitable period of time, the pH is adjusted to approximately 5.0 and the sample is subjected to ion exchange chromatography to produce an eluate. In certain aspects, the ion exchange eluate is collected and further subjected to hydrophobic interactive chromatography to produce an eluate. The hydrophobic interactive chromatography eluate can then be collected for further processing or use.

In certain embodiments the present invention provides for a method of purifying IL-18 antibodies that comprises a primary recovery step to, among other things, remove cells and cellular debris. In certain embodiments of the above-described method, the primary recovery step includes one or more centrifugation or depth filtration steps. For example, and not by way of limitation, such centrifugation steps can be performed at approximately 7000×g to approximately 11,000×g. In addition, certain embodiments of the above-described method will include a depth filtration step, such as a delipid depth filtration step.

In certain embodiments of the above-described method, the ion exchange step can be either cation or anion exchange chromatography, or a combination of both. This step can include multiple ion exchange steps such as a cation exchange step followed by an anion exchange step or visa versa. In certain aspects, the ion exchange step involves a two step ion exchange process. Such two step processes can be accomplished, for example, and not by way of limitation, by a first cation exchange step, followed by a second anion exchange step. An exemplary cation exchange column is a column whose stationary phase comprises anionic groups, such as a CM Hyper DF™ column. This ion exchange capture chromatography step facilitates the isolation of the anti-IL-18 antibodies from the primary recovery mixture. A suitable anion exchange column is a column whose stationary phase comprises cationic groups. An example of such a column is a Q Sepharose™ column. One or more ion exchange step further isolates anti-IL-18 antibodies by reducing impurities such as host cell proteins and DNA and, where applicable, affinity matrix protein. This anion exchange procedure is a flow through mode of chromatography wherein the anti-IL-18 antibodies do not interact or bind to the anion exchange resin (or solid phase). However, many impurities do interact with and bind to the anion exchange resin.

In certain embodiments, a first and second ion exchange step is performed following primary recovery. In certain of such embodiments, the ion exchange sample is subjected to an intermediate filtration step, either prior to the first ion exchange step, between the two ion exchange steps, or both. In certain aspects, this filtration step comprises capture ultrafiltration/diafiltration (“UF/DF”). Among other activities, such filtration facilitates the concentration and buffer exchange of anti-IL-18 antibodies and antigen-binding portions thereof.

Certain embodiments of the invention provide for a method comprising one or more hydrophobic interactive chromatography (“HIC”) step. A suitable HIC column is one whose stationary phase comprises hydrophobic groups. A non-limiting example of such a column is a Phenyl HP Sepharose™ column. In certain circumstances anti-IL-18 antibodies will form aggregates during the isolation/purification process. Inclusion of one or more HIC step facilitates the reduction or elimination of such aggregations. HIC also assists in the removal of impurities. In certain embodiments the HIC step employs a high salt buffer to promote interaction of the anti-IL-18 antibodies (or aggregations thereof) with the hydrophobic column. The anti-IL-18 antibodies can then be eluted using lower concentrations of salt.

In certain embodiments, the HIC eluate is filtered using a viral removal filter such as, but not limited to, an Ultipor DV50™ filter (Pall Corporation, East Hills, N.Y.). Alternative filters, such as Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, Ill.), can also be used in such embodiments.

In certain embodiments, the invention is directed to one or more pharmaceutical composition comprising an isolated anti-IL-18 antibody or antigen-binding portion thereof and an acceptable carrier. In one aspect, the composition further comprises one or more antibody or antigen-binding portion thereof in addition to the anti-IL-18 antibody. In another aspect, the compositions further comprise one or more pharmaceutical agents.

### BRIEF DESCRIPTION OF THE DRAWINGS

FIG. 1 depicts a non-limiting example of a purification scheme of the instant invention.

FIG. 2 discloses the heavy and light chain sequences of a non-limiting example of an anti-IL-18 antibody (ABT-325).

### DETAILED DESCRIPTION OF THE INVENTION

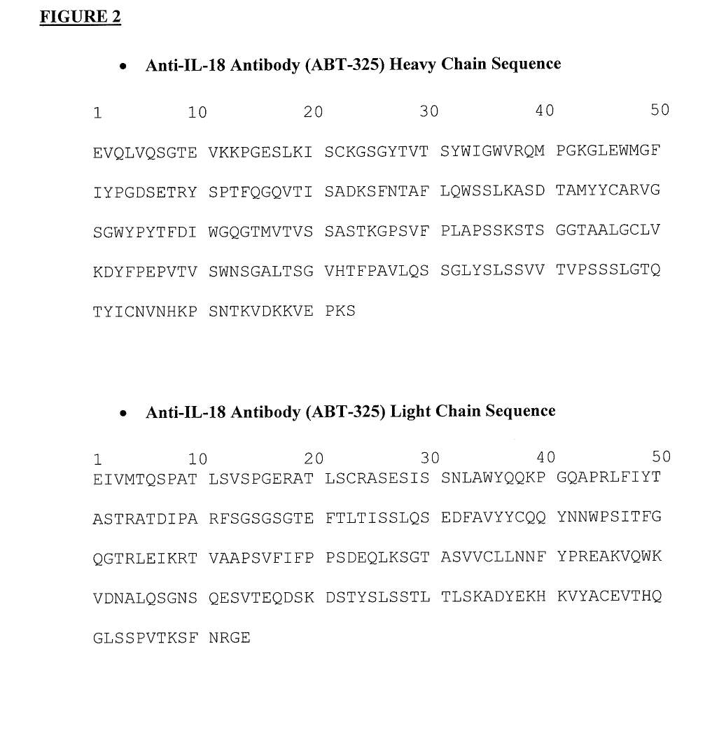

The present invention is directed to antibodies that bind to IL-18. In one aspect, the invention pertains to isolated antibodies, or antigen-binding portions thereof, that bind to human IL-18. The isolated anti-IL-18 antibody of the present invention can be used in a clinical setting as well as in research and development. The present invention also pertains to methods for purifying anti-IL-18 antibodies, or antigen-binding portions thereof. Suitable anti-IL-18 antibodies that may be purified in the context of the instant invention are disclosed in U.S. Ser. Nos. 09/780,035 and 10/988,360, including, the antibody that has subsequently been identified as ABT-325. The heavy and light sequences of ABT-325 are set forth in FIG. 2. The present invention also relates to pharmaceutical compositions comprising the anti-IL-18 antibodies or antigen-binding portions thereof described herein.

For clarity and not by way of limitation, this detailed description is divided into the following sub-portions:

1. Definitions;

2. Antibody Generation;

3. Antibody Production;

4. Antibody Purification;

5. Methods of Assaying Sample Purity;

6. Further Modifications;

7. Pharmaceutical Compositions; and

8. Antibody Uses.

### 1. Definitions

In order that the present invention may be more readily understood, certain terms are first defined.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or “antibody portion”) includes fragments of an antibody that retain the ability to specifically bind to an antigen (e.g., hIL-18). It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CH1 domains; (ii) a F(ab′)₂ fragment, a bivalent fragment comprising two Fab fragments linked by a disulfide bridge at the hinge region; (iii) a Fd fragment comprising the VH and CH1 domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The phrase “human interleukin 18” (abbreviated herein as hIL-18, or IL-18), as used herein, includes a human cytokine that is initially synthesized as biologically inactive 193 amino acid precursor protein as well as the 156 amino acid mature protein produced by, for example, but not by way of limitation, cleavage of the precursor protein, e.g., by caspase-1 or caspase-4, which exhibits biological activities that include the co-stimulation of T cell proliferation, the enhancement of NK cell cytotoxicity, the induction of IFN-γ production by T cells and NK cells, and the potentiation of T helper type 1 (Th1) differentiation. The nucleic acid encoding IL-18 is available as GenBank Accession No. NM_{—}001562 and the polypeptide sequence is available as GenBank Accession No. NP_{—}001553. The term human IL-18 is intended to include recombinant human IL-18 (rh IL-18), which can be prepared by standard recombinant expression methods.

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242, the entire teachings of which are incorporated herein by reference). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “selective mutagenesis approach” includes a method of improving the activity of an antibody by selecting and individually mutating CDR amino acids at least one suitable selective mutagenesis position, hypermutation, and/or contact position. A “selectively mutated” human antibody is an antibody which comprises a mutation at a position selected using a selective mutagenesis approach. In another aspect, the selective mutagenesis approach is intended to provide a method of preferentially mutating selected individual amino acid residues in the CDR1, CDR2 or CDR3 of the heavy chain variable region (hereinafter H1, H2, and H3, respectively), or the CDR1, CDR2 or CDR3 of the light chain variable region (hereinafter referred to as L1, L2, and L3, respectively) of an antibody. Amino acid residues may be selected from selective mutagenesis positions, contact positions, or hypermutation positions. Individual amino acids are selected based on their position in the light or heavy chain variable region. It should be understood that a hypermutation position can also be a contact position. In one aspect, the selective mutagenesis approach is a “targeted approach”. The language “targeted approach” is intended to include a method of mutating selected individual amino acid residues in the CDR1, CDR2 or CDR3 of the heavy chain variable region or the CDR1, CDR2 or CDR3 of the light chain variable region of an antibody in a targeted manner, e.g., a “Group-wise targeted approach” or “CDR-wise targeted approach”. In the “Group-wise targeted approach”, individual amino acid residues in particular groups are targeted for selective mutations including groups I (including L3 and H3), II (including H2 and L1) and III (including L2 and H1), the groups being listed in order of preference for targeting. In the “CDR-wise targeted approach”, individual amino acid residues in particular CDRs are targeted for selective mutations with the order of preference for targeting as follows: H3, L3, H2, L1, H1 and L2. The selected amino acid residue is mutated, e.g., to at least two other amino acid residues, and the effect of the mutation on the activity of the antibody is determined. Activity is measured as a change in the binding specificity/affinity of the antibody, and/or neutralization potency of the antibody. It should be understood that the selective mutagenesis approach can be used for the optimization of any antibody derived from any source including phage display, transgenic animals with human IgG germline genes, human antibodies isolated from human B-cells. The selective mutagenesis approach can be used on antibodies which can not be optimized further using phage display technology. It should be understood that antibodies from any source including phage display, transgenic animals with human IgG germline genes, human antibodies isolated from human B-cells can be subject to back-mutation prior to or after the selective mutagenesis approach.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody” includes an antibody that is substantially free of other antibodies having different antigenic specificities (e.g., an isolated antibody that specifically binds hIL-18 is substantially free of antibodies that specifically bind antigens other than hIL-18). An isolated antibody that specifically binds hIL-18 may bind IL-18 molecules from other species. Moreover, an isolated antibody may be substantially free of other cellular material and/or chemicals.

A “neutralizing antibody” (or an “antibody that neutralized hIL-18 activity”) includes an antibody whose binding to hIL-18 results in inhibition of the biological activity of hIL-18. This inhibition of the biological activity of hIL-18 can be assessed by measuring one or more indicators of hIL-18 biological activity, such as induction of IFNγ production by T cells or NK cells, or inhibition of IL-18 receptor binding in a human IL-18 receptor binding assay. These indicators of hIL-18 biological activity can be assessed by one or more of several standard in vitro or in vivo assays known in the art.

The term “activity” includes activities such as the binding specificity/affinity of an antibody for an antigen, e.g., an anti-hIL-18 antibody that binds to an IL-18 antigen and/or the neutralizing potency of an antibody, e.g., an anti-hIL-18 antibody whose binding to hIL-18 inhibits the biological activity of hIL-18, e.g., inhibition of PHA blast proliferation or inhibition of receptor binding in a human IL-18 receptor binding assay.

The phrase “surface plasmon resonance” includes an optical phenomenon that allows for the analysis of real-time biospecific interactions by detection of alterations in protein concentrations within a biosensor matrix, e.g., using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277, the entire teachings of which are incorporated herein.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The phrase “nucleic acid molecule” includes DNA molecules and RNA molecules. A nucleic acid molecule may be single-stranded or double-stranded, but in one aspect is double-stranded DNA.

The phrase “isolated nucleic acid molecule,” as used herein in reference to nucleic acids encoding antibodies or antibody portions (e.g., VH, VL, CDR3) that bind hIL-18 (including “isolated antibodies”), includes a nucleic acid molecule in which the nucleotide sequences encoding the antibody or antibody portion are free of other nucleotide sequences encoding antibodies or antibody portions that bind antigens other than hIL-18, which other sequences may naturally flank the nucleic acid in human genomic DNA. Thus, e.g., an isolated nucleic acid of the invention encoding a VH region of an anti-IL-18 antibody contains no other sequences encoding other VH regions that bind antigens other than IL-18. The phrase “isolated nucleic acid molecule” is also intended to include sequences encoding bivalent, bispecific antibodies, such as diabodies in which VH and VL regions contain no other sequences other than the sequences of the diabody.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “modifying”, as used herein, is intended to refer to changing one or more amino acids in the antibodies or antigen-binding portions thereof. The change can be produced by adding, substituting or deleting an amino acid at one or more positions. The change can be produced using known techniques, such as PCR mutagenesis.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The phrase “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”). Such decreases in the number and/or activity of viral particles can be on the order of about 1% to about 99%, preferably of about 20% to about 99%, more preferably of about 30% to about 99%, more preferably of about 40% to about 99%, even more preferably of about 50% to about 99%, even more preferably of about 60% to about 99%, yet more preferably of about 70% to about 99%, yet more preferably of about 80% to 99%, and yet more preferably of about 90% to about 99%. In certain non-limiting embodiments, the amount of virus, if any, in the purified antibody product is less than the ID50 (the amount of virus that will infect 50 percent of a target population) for that virus, preferably at least 10-fold less than the ID50 for that virus, more preferably at least 100-fold less than the ID50 for that virus, and still more preferably at least 1000-fold less than the ID50 for that virus.

The phrase “contact position” includes an amino acid position in the CDR1, CDR2 or CDR3 of the heavy chain variable region or the light chain variable region of an antibody which is occupied by an amino acid that contacts antigen in one of the twenty-six known antibody-antigen structures. If a CDR amino acid in any of the twenty-six known solved structures of antibody-antigen complexes contacts the antigen, then that amino acid can be considered to occupy a contact position. Contact positions have a higher probability of being occupied by an amino acid which contact antigens than in a non-contact position. In one aspect, a contact position is a CDR position which contains an amino acid that contacts antigen in greater than 3 of the 26 structures (>1.5%). In another aspect, a contact position is a CDR position which contains an amino acid that contacts antigen in greater than 8 of the 25 structures (>32%).

### 2. Antibody Generation

The term “antibody” as used in this section refers to an intact antibody or an antigen binding fragment thereof.

The antibodies of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

One preferred animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody preferably can be a human, a chimeric, or a humanized antibody. Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In one non-limiting embodiment, the antibodies of this disclosure are human monoclonal antibodies. Such human monoclonal antibodies directed against IL-18 can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen).

Moreover, alternative transchromosomic animal systems expressing human immunoglobulin genes are available in the art and can be used to raise anti-IL-18 antibodies of this disclosure. For example, mice carrying both a human heavy chain transchromosome and a human light chain transchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise anti-IL-18 antibodies of this disclosure.

Recombinant human antibodies of the invention, including anti-IL-18 antibodies or an antigen binding portion thereof, or anti-IL-18-related antibodies disclosed herein can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In one embodiment, the methods of the invention include anti-IL-18 antibodies and antibody portions, anti-IL-18-related antibodies and antibody portions, and human antibodies and antibody portions with equivalent properties to anti-IL-18 antibodies, such as high affinity binding to hIL-18 with low dissociation kinetics and high neutralizing capacity. In one aspect, the invention provides treatment with an isolated human antibody, or an antigen-binding portion thereof, that dissociates from hIL-18 with a K_{d} of about 1×10⁻⁸ M or less and a K_{off} rate constant of 1×10⁻³ s⁻¹ or less, both determined by surface plasmon resonance. In specific non-limiting embodiments, an anti-IL-18 antibody purified according to the invention competitively inhibits binding of ABT-325 to IL-18 under physiological conditions.

In yet another embodiment of the invention, anti-IL-18 antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the antibody may also reduce other effector functions which rely on FcR interactions, such as opsonization and phagocytosis and antigen-dependent cellular cytotoxicity.

### 3. Antibody Production

To express an antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Prior to insertion of the antibody or antibody-related light or heavy chain sequences, the expression vector may already carry antibody constant region sequences. For example, one approach to converting the anti-IL-18 antibody or anti-IL-18 antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, a recombinant expression vector of the invention can carry one or more regulatory sequence that controls the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

In addition to the antibody chain genes and regulatory sequences, a recombinant expression vector of the invention may carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr⁻ host cells with methotrexate selection/amplification) and the neo gene (for G418 selection).

An antibody, or antibody portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to IL-18, specifically hIL-18. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than IL-18 by crosslinking an antibody of the invention to a second antibody by standard chemical crosslinking methods.

In a suitable system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

When using recombinant techniques, the antibody can be produced intracellularly, in the periplasmic space, or directly secreted into the medium. In one aspect, if the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon or Millipore Pellicon ultrafiltration unit.

Prior to the process of the invention, procedures for purification of antibodies from cell debris initially depend on the site of expression of the antibody. Some antibodies can be secreted directly from the cell into the surrounding growth media; others are made intracellularly. For the latter antibodies, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon or Millipore Pellicon ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

### 4. Antibody Purification

4.1 Antibody Purification Generally

The invention provides a method for producing a purified (or “HCP-reduced”) antibody preparation from a mixture comprising an antibody and at least one HCP. The purification process of the invention begins at the separation step when the antibody has been produced using methods described above and conventional methods in the art. Typically in the art, antibody-HCP mixtures are subjected to protein A capture (e.g., a protein A column) as an initial purification step, since the antibody binds to protein A whereas HCP will flow through. The purification methods of the present invention have the advantage that it is not necessary to subject the mixture comprising an antibody and at least one HCP to protein A capture (e.g., a protein A column) as an initial step, or as any step in the purification method. Table 1 summarizes one embodiment of a purification scheme. Variations of this scheme are envisaged and are within the scope of this invention.

TABLE 1
Purification steps with their associated purpose

| Purification step | Purpose |
|---|---|
| Primary recovery | clarification of sample matrix |
| Cation exchange | antibody capture, host cell protein |
| chromatography | and associated impurity reduction |
| ultrafiltration/diafiltration | concentration and buffer exchange |
| Anion exchange | reduction of host cell proteins and DNA |
| chromatography | |
| Phenyl Sepharose HP | reduction of antibody aggregates |
| chromatography | and host cell proteins |
| Viral filtration | removal of large viruses, if present |
| Final ultrafiltration/diafiltration | concentrate and formulate antibody |

Once a clarified solution or mixture comprising the antibody has been obtained, separation of the antibody from the other proteins produced by the cell, such as HCPs, is performed using a combination of different purification techniques, including ion exchange separation step(s) and hydrophobic interaction separation step(s). The separation steps separate mixtures of proteins on the basis of their charge, degree of hydrophobicity, or size. In one aspect of the invention, separation is performed using chromatography, including cationic, anionic, and hydrophobic interaction. Several different chromatography resins are available for each of these techniques, allowing accurate tailoring of the purification scheme to the particular protein involved. The essence of each of the separation methods is that proteins can be caused either to traverse at different rates down a column, achieving a physical separation that increases as they pass further down the column, or to adhere selectively to the separation medium, being then differentially eluted by different solvents. In some cases, the antibody is separated from impurities when the impurities specifically adhere to the column and the antibody does not, i.e., the antibody is present in the flow through.

As noted above, accurate tailoring of a purification scheme relies on consideration of the protein to be purified. In certain embodiments, the separation steps of the instant invention are employed to separate an antibody from one or more HCPs. Antibodies that can be successfully purified using the methods described herein include, but are not limited to, human IgA₁, IgA₂, IgD, IgE, IgG₁, IgG₂, IgG₃, IgG₄, and IgM antibodies. In certain embodiments, the purification strategies of the instant invention exclude the use of Protein A affinity chromatography. Such embodiments are particularly useful for the purification of IgG₃ antibodies, as IgG₃ antibodies are known to bind to Protein A inefficiently. Other factors that allow for specific tailoring of a purification scheme include, but are not limited to: the presence or absence of an Fc region (e.g., in the context of full length antibody as compared to an Fab fragment thereof); the particular germline sequences employed in generating to antibody of interest; and the amino acid composition of the antibody (e.g., the primary sequence of the antibody as well as the overall charge/hydrophobicity of the molecule). Antibodies sharing one or more characteristic can be purified using purification strategies tailored to take advantage of that characteristic.

4.2 Primary Recovery

The initial steps of the purification methods of the present invention involve the first phase of clarification and primary recovery of anti-IL-18 antibody from a sample matrix. In addition, the primary recovery process can also be a point at which to inactivate viruses that can be present in the sample matrix. For example, any one or more of a variety of methods of viral inactivation can be used during the primary recovery phase of purification including heat inactivation (pasteurization), pH inactivation, solvent/detergent treatment, UV and γ-ray irradiation and the addition of certain chemical inactivating agents such as β-propiolactone or e.g., copper phenanthroline as in U.S. Pat. No. 4,534,972, the entire teaching of which is incorporated herein by reference. In certain embodiments of the present invention, the sample matrix is exposed to pH viral inactivation during the primary recovery phase.

Methods of pH viral inactivation include, but are not limited to, incubating the mixture for a period of time at low pH, and subsequently neutralizing the pH and removing particulates by filtration. In certain embodiments the mixture will be incubated at a pH of 2 to 5, preferably at a pH of 3 to 4, and more preferably at a pH of 3.5. The pH of the sample mixture may be lowered by any suitable acid including, but not limited to, citric acid, acetic acid, caprylic acid, or other suitable acids. The choice of pH level largely depends on the stability profile of the antibody product and buffer components. It is known that the quality of the target antibody during low pH virus inactivation is affected by pH and the duration of the low pH incubation. In certain embodiments the duration of the low pH incubation will be from 0.5 hr to two 2 hr, preferably 0.5 hr to 1.5 hr, and more preferably the duration will be 1 hr. Virus inactivation is dependent on these same parameters in addition to protein concentration, which may reduce inactivation at high concentrations. Thus, the proper parameters of protein concentration, pH, and duration of inactivation can be selected to achieve the desired level of viral inactivation.

In certain embodiments viral inactivation can be achieved via the use of suitable filters. A non-limiting example of a suitable filter is the Ultipor DV50™ filter from Pall Corporation. Although certain embodiments of the present invention employ such filtration during the primary recovery phase, in other embodiments it is employed at other phases of the purification process, including as either the penultimate or final step of purification. In certain embodiments, alternative filters are employed for viral inactivation, such as, but not limited to, Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, Ill.).

In those embodiments where viral inactivation is employed, the sample mixture can be adjusted, as needed, for further purification steps. For example, following low pH viral inactivation the pH of the sample mixture is typically adjusted to a more neutral pH, e.g., from about 5.0 to about 8.5 prior to continuing the purification process. Additionally, the mixture may be flushed with water for injection (WFI) to obtain a desired conductivity.

In certain embodiments, the primary recovery will include one or more centrifugation steps to further clarify the sample matrix and thereby aid in purifying the anti-IL-18 antibodies. Centrifugation of the sample can be run at, for example, but not by way of limitation, 7,000×g to approximately 12,750×g. In the context of large scale purification, such centrifugation can occur on-line with a flow rate set to achieve, for example, but not by way of limitation, a turbidity level of 150 NTU in the resulting supernatant. Such supernatant can then be collected for further purification.

In certain embodiments, the primary recovery will include the use of one or more depth filtration steps to further clarify the sample matrix and thereby aid in purifying the anti-IL-18 antibodies. Depth filters contain filtration media having a graded density. Such graded density allows larger particles to be trapped near the surface of the filter while smaller particles penetrate the larger open areas at the surface of the filter, only to be trapped in the smaller openings nearer to the center of the filter. In certain embodiments the depth filtration step can be a delipid depth filtration step. Although certain embodiments employ depth filtration steps only during the primary recovery phase, other embodiments employ depth filters, including delipid depth filters, during one or more additional phases of purification. Non-limiting examples of depth filters that can be used in the context of the instant invention include the Cuno™ model 30/60ZA depth filters (3M Corp.), and 0.45/0.2 μm Sartopore™ bi-layer filter cartridges.

4.3 Ion Exchange Chromatography

In certain embodiments, the instant invention provides methods for producing a HCP-reduced antibody preparation from a mixture comprising an antibody and at least one HCP by subjecting the mixture to at least one ion exchange separation step such that an eluate comprising the antibody is obtained. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein. Therefore, it is within the scope of this invention to employ an anionic exchange step prior to the use of a cationic exchange step, or a cationic exchange step prior to the use of an anionic exchange step. Furthermore, it is within the scope of this invention to employ only a cationic exchange step, only an anionic exchange step, or any serial combination of the two.

In performing the separation, the initial antibody mixture can be contacted with the ion exchange material by using any of a variety of techniques, e.g., using a batch purification technique or a chromatographic technique.

For example, in the context of batch purification, ion exchange material is prepared in, or equilibrated to, the desired starting buffer. Upon preparation, or equilibration, a slurry of the ion exchange material is obtained. The antibody solution is contacted with the slurry to adsorb the antibody to be separated to the ion exchange material. The solution comprising the HCP(s) that do not bind to the ion exchange material is separated from the slurry, e.g., by allowing the slurry to settle and removing the supernatant. The slurry can be subjected to one or more wash steps. If desired, the slurry can be contacted with a solution of higher conductivity to desorb HCPs that have bound to the ion exchange material. In order to elute bound polypeptides, the salt concentration of the buffer can be increased.

Ion exchange chromatography may also be used as an ion exchange separation technique. Ion exchange chromatography separates molecules based on differences between the overall charge of the molecules. For the purification of an antibody, the antibody must have a charge opposite to that of the functional group attached to the ion exchange material, e.g., resin, in order to bind. For example, antibodies, which generally have an overall positive charge in the buffer pH below its pI, will bind well to cation exchange material, which contain negatively charged functional groups.

In ion exchange chromatography, charged patches on the surface of the solute are attracted by opposite charges attached to a chromatography matrix, provided the ionic strength of the surrounding buffer is low. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution).

Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substitutents include carboxymethyl (CM), sulfoethyl (SE), sulfopropyl (SP), phosphate (P) and sulfonate (S). Cellulose ion exchange resins such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow are all available from Pharmacia AB. Further, both DEAE and CM derivatized ethylene glycol-methacrylate copolymer such as TOYOPEARL™ DEAE-650S or M and TOYOPEARL™ CM-650S or M are available from Toso Haas Co., Philadelphia, Pa.

A mixture comprising an antibody and impurities, e.g., HCP(s), is loaded onto an ion exchange column, such as a cation exchange column. For example, but not by way of limitation, the mixture can be loaded at a load of about 80 g protein/L resin depending upon the column used. An example of a suitable cation exchange column is a 80 cm diameter×23 cm long column whose bed volume is about 116 L. The mixture loaded onto this cation column can subsequently washed with wash buffer (equilibration buffer). The antibody is then eluted from the column, and a first eluate is obtained.

This ion exchange step facilitates the capture of the antibody of interest while reducing impurities such as HCPs. In certain aspects, the ion exchange column is a cation exchange column. For example, but not by way of limitation, a suitable resin for such a cation exchange column is CM HyperDF resin. These resins are available from commercial sources such as Pall Corporation. This cation exchange procedure can be carried out at or around room temperature.

4.4 Ultrafiltration/Diafiltration

Certain embodiments of the present invention employ ultrafiltration and/or diafiltration steps to further purify and concentration the anti-IL-18 antibody sample, Ultrafiltration is described in detail in, Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally referred to filtration using filters with a pore size of smaller than 0.1 μm. By employing filters having such small pore size, the volume of the sample can be reduced through permeation of the sample buffer through the filter while the anti-IL-18 antibodies is be retained.

Diafiltration is a method of using ultrafilters to remove and exchange salts, sugars, non-aqueous solvents, separation of free from bound species, removal of material of low molecular weight, or cause the rapid change of ionic and/or pH environments. Such microsolutes are removed most efficiently by adding solvent to the solution being ultrafiltered at a rate equal to the ultrafiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody preparations.

4.5 Hydrophobic Interaction Chromatography

The present invention also features methods for producing a HCP-reduced antibody preparation from a mixture comprising an antibody and at least one HCP further comprising a hydrophobic interaction separation step. For example, a first eluate obtained from an ion exchange column can be subjected to a hydrophobic interaction material such that a second eluate having a reduced level of HCP is obtained. Hydrophobic interaction chromatography steps, such as those disclosed herein, are generally performed to remove protein aggregates, such as antibody aggregates, and process-related impurities.

In performing the separation, the sample mixture is contacted with the HIC material, e.g., using a batch purification technique or using a column. Prior to HIC purification it may be desirable to remove any chaotropic agents or very hydrophobic substances, e.g., by passing the mixture through a pre-column.

For example, in the context of batch purification, HIC material is prepared in or equilibrated to the desired equilibration buffer. A slurry of the HIC material is obtained. The antibody solution is contacted with the slurry to adsorb the antibody to be separated to the HIC material. The solution comprising the HCPs that do not bind to the HIC material is separated from the slurry, e.g., by allowing the slurry to settle and removing the supernatant. The slurry can be subjected to one or more washing steps. If desired, the slurry can be contacted with a solution of lower conductivity to desorb antibodies that have bound to the HIC material. In order to elute bound antibodies, the salt concentration can be decreased.

Whereas ion exchange chromatography relies on the charges of the antibodies to isolate them, hydrophobic interaction chromatography uses the hydrophobic properties of the antibodies. Hydrophobic groups on the antibody interact with hydrophobic groups on the column. The more hydrophobic a protein is the stronger it will interact with the column. Thus the HIC step removes host cell derived impurities (e.g., DNA and other high and low molecular weight product-related species).

Hydrophobic interactions are strongest at high ionic strength, therefore, this form of separation is conveniently performed following salt precipitations or ion exchange procedures. Adsorption of the antibody to a HIC column is favored by high salt concentrations, but the actual concentrations can vary over a wide range depending on the nature of the antibody and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba⁺⁺; Ca⁺⁺; Mg⁺⁺; Li⁺; Cs⁺; Na⁺; K⁺; Rb⁺; NH₄⁺, while anions may be ranked in terms of increasing chaotropic effect as P0⁻⁻⁻; S0₄⁻⁻; CH₃CO₃⁻; Cl⁻; Br⁻; NO₃⁻; ClO₄⁻; I⁻; SCN⁻.

In general, Na, K or NH₄ sulfates effectively promote ligand-protein interaction in HIC. Salts may be formulated that influence the strength of the interaction as given by the following relationship: (NH₄)₂SO₄>Na₂SO₄>NaCl>NH₄Cl>NaBr>NaSCN. In general, salt concentrations of between about 0.75 and about 2 M ammonium sulfate or between about 1 and 4 M NaCl are useful.

HIC columns normally comprise a base matrix (e.g., cross-linked agarose or synthetic copolymer material) to which hydrobobic ligands (e.g., alkyl or aryl groups) are coupled. A suitable HIC column comprises an agarose resin substituted with phenyl groups (e.g., a Phenyl Sepharose™ column). Many HIC columns are available commercially. Examples include, but are not limited to, Phenyl Sepharose™ 6 Fast Flow column with low or high substitution (Pharmacia LKB Biotechnology, AB, Sweden); Phenyl Sepharose™ High Performance column (Pharmacia LKB Biotechnology, AB, Sweden); Octyl Sepharose™ High Performance column (Pharmacia LKB Biotechnology, AB, Sweden); Fractogel™ EMD Propyl or Fractogel™ EMD Phenyl columns (E. Merck, Germany); Macro-Prep™ Methyl or Macro-Prep™ t-Butyl Supports (Bio-Rad, California); WP HI-Propyl (C₃)™ column (J. T. Baker, New Jersey); and Toyopearl™ ether, phenyl or butyl columns (TosoHaas, PA)

4.6 Exemplary Purification Strategies

In certain embodiments, primary recovery can proceed by sequentially employing pH reduction, centrifugation, and filtration steps to remove cells and cell debris (including HCPs) from a production bioreactor harvest. For example, but not by way of limitation, such primary recovery can be accomplished first by removal of host cells by centrifugation (6900×g) and pH reduction, with final clarification by centrifugation (12750×g) and depth filtration. In certain embodiments the culture comprising antibodies and media can be subjected to pH inactivation using a pH of about 3.5 to about 4.0 for approximately 1 to 1.5 hours at about 20° C. The pH reduction can be facilitated using known acid preparations such as citric acid, e.g., 3 M citric acid, phosphoric acid, acetic acid, formic acid and the like. This pH reduction reduces/inactivates, if not completely eliminates, pH sensitive virus contaminants and precipitates some media and host cell contaminants. Following this reduction the acidified harvest pH can be adjusted to about 4.5 to about 5.5 using a base such as sodium hydroxide, e.g., 3 M sodium hydroxide, and held for about 16-24 hours at about 8° C. Following the 16-24 hour period, the temperature can be brought to around 20° C. The pH adjusted culture can be centrifuged at around 12,750×g. The resulting sample supernatant can then be passed through a filter train comprising, e.g., one 3×12″ filter housing fitted with three 12-inch Cuno™ model 60ZA depth filters of nominal pore sizes ranging from about 0.2 to about 0.8 μm and one 3×30″ filter housing fitted with three 30″-0.22 μm hydrophobic filter cartridges. Other suitable filter systems are commercially available and are within the scope of the invention. It should be noted that one skilled in the art may vary the conditions recited above and still be within the scope of the present invention.

In certain embodiments, the clarified supernatant is then further purified using cation exchange column. In certain aspects, the equilibrating buffer is a buffer having a pH of about 5.0. A non-limiting example of a suitable buffer is about 20 mM sodium citrate/citric acid with 65 mM NaCl, pH 5.0. Following equilibration, the column is loaded with sample prepared from the primary recovery step above. The column is then washed using the equilibrating buffer. The column is next subjected to an elution step using a buffer having a greater ionic strength as compared to the equilibrating buffer. For example, a suitable elution buffer can be about 20 mM sodium citrate/citric acid, 300 mM NaCl, pH 5.0. The anti-IL-18 antibodies will be eluted and can be monitored using a UV spectrophotometer set at OD_{280 nm}. In a particular example, the column eluate can be collected as the absorbance rises above 3 OD_{280 nm} and continue until approximately to 2 OD_{280 nm}. It should be understood that one skilled in the art may vary the conditions and yet still be within the scope of the invention.

In certain embodiments, the cation exchange eluate is next filtered using, e.g., a 30 kD MW cutoff filter. A suitable filter for this filtering step is, e.g., Millipore's 30 kD molecular weight cut-off (MWCO) cellulose ultrafilter membrane cassette. Ultrafiltration can continue until the eluate reaches a final target concentration of, e.g., 30 mg/mL. This filtrate can then be diafiltered using an appropriate buffer. An example of an appropriated buffer is 20 mM sodium phosphate and 150 mM sodium chloride, pH around 7.0.

In certain embodiments, the sample from the capture filtration step above is subjected to a second ion exchange separation, such as an anion exchange chromatographic step. Alternatively, the cation exchange elute can be subjected to anion exchange chromatography where the cation exchange elute is equilibrated to the appropriate buffer. This anion exchange step reduces process related impurities such as nucleic acids like host cell proteins and DNA. This ion exchange step is a flow through mode of chromatography where the antibodies of interest do not interact with nor bind to the solid phase of the column, e.g., to the Q Sepharose™. However, many impurities will in fact interact with and bind to the column's solid phase. The anion exchange can be performed at about 12° C.

A non-limiting example of a suitable column for this step is one packed with an anion exchange resin such as Q Sepharose™ Fast Flow from GE Healthcare, Piscataway, N.J. The column can be equilibrated using multiple (e.g., about 5-7) column volumes of an appropriate buffer such as trolamine/sodium chloride. An example of suitable conditions include about 25 mM trolamine with about 40 mM sodium chloride at pH 8.0. Again, a skill artisan may vary the conditions but still be within the scope of the present invention. The collected sample from UF/DF step outlined above is diluted with two volumes of 50 mM trolamine, pH 8 and loaded onto the anion exchange column. In alternative embodiments, the column is loaded from the eluate collected during cation exchange after pH and conductivity adjustments. Following the loading of the column, the column is washed with the equilibration buffer. The flow-through comprising the anti-IL-18 antibodies can be monitored using a UV spectrophotometer at OD_{280 nm}. In certain examples, elution collection can be from upside 0.4 OD_{280 nm} to downside 0.6 OD_{280 nm}.

The present invention also features methods for producing a HCP-reduced antibody preparation from a mixture comprising an antibody and at least one HCP further comprising a hydrophobic interaction separation step wherein the ion exchange flow-through is subjected to a hydrophobic interaction material such that a second eluate having a reduced level of HCP is obtained.

In performing the separation, the sample mixture is contacted with the HIC material, e.g., using a batch purification technique or using a column. Prior to HIC purification it may be desirable to remove any chaotropic agents or very hydrophobic substances. As an example, for batch purification, HIC material is prepared in or equilibrated to the desired equilibration buffer. A slurry of the HIC material is obtained. The antibody solution is contacted with the slurry to adsorb the antibody to be separated to the HIC material. The solution comprising the HCPs that do not bind to the HIC material is separated from the slurry, e.g., by allowing the slurry to settle and removing the supernatant. The slurry can be subjected to one or more washing steps. If desired, the slurry can be contacted with a solution of lower conductivity to desorb antibodies that have bound to the HIC material. In order to elute bound antibodies, the salt concentration can be decreased.

In certain embodiments of the invention, the sample containing anti-IL-18 antibodies will be further processed using a hydrophobic interaction separation step. In certain embodiments the hydrophobic interaction separation step will include a hydrophobic interaction chromatography (HIC) step. A non-limiting example of a suitable column for the HIC step is one packed with and HIC resin, such as Phenyl HP Sepharose™ from GE Healthcare Pharmacia, Piscataway, N.J. The flow-through preparation obtained from the previous step comprising the antibodies of interest can be diluted with an equal volume of around 2.2 M ammonium sulfate, 40 mM sodium phosphate, pH 7.0. This then can be subjected to filtration using about a 0.45/0.2 μm Sartopore™ 2 bi-layer filter, or its equivalent. In certain embodiments, the hydrophobic chromatography procedure involves two or more cycles.

In certain embodiments, the HIC column is first equilibrated using a suitable buffer. An example of a suitable buffer is 1.1 M ammonium sulfate, 20 mM sodium phosphate, pH 7.0. One skilled in the art can vary the equilibrating buffer and still be within the scope of the present invention by altering the concentrations of the buffering agents and/or by substituting equivalent buffers. The column is loaded with the diluted anion exchange flow-through sample and washed multiple times, e.g., three times, with equilibration buffer.

The column is eluted using an appropriate elution buffer. A suitable example of such an elution buffer is 0.3 M ammonium sulfate, 9 mM sodium phosphate at a pH around 7.0. The antibodies of interest can be detected and collected using a conventional spectrophotometer from the upside at 1 OD_{280 nm} to downside of peak at 4 OD_{280 nm}.

In certain embodiments of the invention, the eluate from the hydrophobic chromatography step is subjected to filtration for the removal of viral particles, including intact viruses. A suitable filter is the Ultipor DV50™ filter from Pall Filtron, Northborough, Mass. Other viral filters can be used in this filtration step and are well known to those skilled in the art. In a particular aspect, the HIC eluate is passed through a pre-wetted filter train consisting of a 0.1 μm filter and a 10 inch Ultipor DV50™ nanofilter at around 34 psig. Optionally, following the filtration process, the filter is washed using, e.g., the HIC elution buffer in order to remove any antibodies retained in the filter housing. The filtrate can be stored in a pre-sterilized container at around 12° C.

In further embodiments, the filtrate from the above is again subjected to ultrafiltration/diafiltration. This step is important if a practitioner's end point is to use the antibody in a, e.g., pharmaceutical formulation. Ultrafiltration facilitates the concentration of antibody, and diafiltration facilitates removal of buffering salts previously used and replace it with a particular formulation buffer. Continuous diafiltration with multiple volumes, e.g., two volumes or more, of a formulation buffer is performed. An example of a suitable formulation buffer is 5 mM methionine, 2% mannitol, 0.5% sucrose, pH 5.9 buffer. Upon completion of diafiltration, the antibody is concentrated. One skilled in the art may wish to further filter the antibody product at this point using methods well known in the art.

Certain embodiments of the present invention will include further purification steps. Examples of additional purification procedures which may be performed prior to, during, or following the ion exchange chromatography method include ethanol precipitation, isoelectric focusing, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxyapatite chromatography, gel electrophoresis, dialysis, and affinity chromatography (e.g., using protein A, protein G, an antibody, a specific substrate, ligand or antigen as the capture reagent).

### 5. Methods of Assaying Sample Purity

The present invention also provides methods for determining the residual levels of host cell protein (HCP) concentration in the isolated/purified antibody composition. As described above, HCPs are desirably excluded from the final target substance product, the anti-IL-18 antibody. Exemplary HCPs include proteins originating from the source of the antibody production. Failure to identify and sufficiently remove HCPs from the target antibody may lead to reduced efficacy and/or adverse subject reactions.

As used herein, the term “HCP ELISA” refers to an ELISA where the second antibody used in the assay is specific to the HCPs produced from cells, e.g., CHO cells, used to generate the antibody, anti-IL-18 antibody. The second antibody may be produced according to conventional methods known to those of skill in the art. For example, the second antibody may be produced using HCPs obtained by sham production and purification runs, i.e., the same cell line used to produce the antibody of interest is used, but the cell line is not transfected with antibody DNA. In an exemplary embodiment, the second antibody is produced using HPCs similar to those expressed in the cell expression system of choice, i.e., the cell expression system used to produce the target antibody.

Generally, HCP ELISA comprises sandwiching a liquid sample comprising HCPs between two layers of antibodies, i.e., a first antibody and a second antibody. The sample is incubated during which time the HCPs in the sample are captured by the first antibody, for example, but not limited to goat anti-CHO, affinity purified (Cygnus). A labeled second antibody, or blend of antibodies, specific to the HCPs produced from the cells used to generate the antibody, e.g., anti-CHO HCP Biotinylated, is added, and binds to the HCPs within the sample. In certain embodiments the first and second antibodies are polyclonal antibodies. In certain aspects the first and second antibodies are blends of polyclonal antibodies raised against HCPs, for example, but not limited to Biotinylated goat anti Host Cell Protein Mixture 599/626/748. The amount of HCP contained in the sample is determined using the appropriate test based on the label of the second antibody.

HCP ELISA may be used for determining the level of HCPs in an antibody composition, such as an eluate or flow-through obtained using the process described in section III above. The present invention also provides a composition comprising an antibody, wherein the composition has no detectable level of HCPs as determined by an HCP Enzyme Linked Immunosorbent Assay (“ELISA”).

### 6. Further Modifications

The anti-IL-18 antibodies of the present invention can be modified. In some embodiments, the anti-IL-18 antibodies or antigen binding fragments thereof, are chemically modified to provide a desired effect. For example, pegylation of antibodies and antibody fragments of the invention may be carried out by any of the pegylation reactions known in the art, as described, e.g., in the following references: Focus on Growth Factors 3:4-10 (1992); EP 0 154 316; and EP 0 401 384, each of which is incorporated by reference herein in its entirety. In one aspect, the pegylation is carried out via an acylation reaction or an alkylation reaction with a reactive polyethylene glycol molecule (or an analogous reactive water-soluble polymer). A suitable water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl-ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under suitable conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat IL-18-related disorders of the invention by administration of the anti-IL-18 antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

An antibody or antibody portion of the invention can be derivatized or linked to another functional molecule (e.g., another peptide or protein). Accordingly, the antibodies and antibody portions of the invention are intended to include derivatized and otherwise modified forms of the human anti-hIL-18 antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

### 7. Pharmaceutical Compositions

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is desirable to include isotonic agents, e.g., sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The antibodies and antibody-portions of the invention can be incorporated into a pharmaceutical composition suitable for parenteral administration. The antibody or antibody-portions can be prepared as an injectable solution containing, e.g., 0.1-250 mg/mL antibody. The injectable solution can be composed of either a liquid or lyophilized dosage form in a flint or amber vial, ampule or pre-filled syringe. The buffer can be L-histidine approximately 1-50 mM, (optimally 5-10 mM), at pH 5.0 to 7.0 (optimally pH 6.0). Other suitable buffers include but are not limited to sodium succinate, sodium citrate, sodium phosphate or potassium phosphate. Sodium chloride can be used to modify the tonicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 24%). Stabilizers can be used in both liquid and lyophilized dosage forms, principally 1-50 mM L-methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

In one aspect, the pharmaceutical composition includes the antibody at a dosage of about 0.01 mg/kg-10 mg/kg. In another aspect, the dosages of the antibody include approximately 1 mg/kg administered every other week, or approximately 0.3 mg/kg administered weekly. A skilled practitioner can ascertain the proper dosage and regime for administering to a subject.

The compositions of this invention may be in a variety of forms. These include, e.g., liquid, semi-solid and solid dosage forms, such as liquid solutions (e.g., injectable and infusible solutions), dispersions or suspensions, tablets, pills, powders, liposomes and suppositories. The form depends on, e.g., the intended mode of administration and therapeutic application. Typical compositions are in the form of injectable or infusible solutions, such as compositions similar to those used for passive immunization of humans with other antibodies. One mode of administration is parenteral (e.g., intravenous, subcutaneous, intraperitoneal, intramuscular). In one aspect, the antibody is administered by intravenous infusion or injection. In another aspect, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the methods of preparation are vacuum drying and spray-drying that yields a powder of the active ingredient plus any additional desired ingredient from a previously sterile-filtered solution thereof. The proper fluidity of a solution can be maintained, e.g., by the use of a coating such as lecithin, by the maintenance of the required particle size in the case of dispersion and by the use of surfactants. Prolonged absorption of injectable compositions can be brought about by including in the composition an agent that delays absorption, e.g., monostearate salts and gelatin.

The antibodies and antibody-portions of the present invention can be administered by a variety of methods known in the art, one route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978, the entire teaching of which is incorporated herein by reference.

In certain aspects, an antibody or antibody portion of the invention may be orally administered, e.g., with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain aspects, an antibody or antibody portion of the invention is co-formulated with and/or co-administered with one or more additional therapeutic agents that are useful for treating disorders in which IL-18 activity is detrimental. For example, an anti-hIL-18 antibody or antibody portion of the invention may be co-formulated and/or co-administered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies. It will be appreciated by the skilled practitioner that when the antibodies of the invention are used as part of a combination therapy, a lower dosage of antibody may be desirable than when the antibody alone is administered to a subject (e.g., a synergistic therapeutic effect may be achieved through the use of combination therapy which, in turn, permits use of a lower dose of the antibody to achieve the desired therapeutic effect).

Antibodies of the invention, or antigen binding portions thereof can be used alone or in combination to treat such diseases. It should be understood that the antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent which imparts a beneficial attribute to the therapeutic composition, e.g., an agent which affects the viscosity of the composition.

It should further be understood that the combinations which are to be included within this invention are those combinations useful for their intended purpose. The agents set forth below are illustrative and not intended to be limited. The combinations which are part of this invention can be the antibodies of the present invention and at least one additional agent selected from the lists below. The combination can also include more than one additional agent, e.g., two or three additional agents if the combination is such that the formed composition can perform its intended function.

Some combinations are non-steroidal anti-inflammatory drug(s) also referred to as NSAIDS which include drugs like ibuprofen. Other combinations are corticosteroids including prednisolone; the well known side-effects of steroid use can be reduced or even eliminated by tapering the steroid dose required when treating patients in combination with the anti-IL-18 antibodies of this invention. Non-limiting examples of therapeutic agents for rheumatoid arthritis with which an antibody, or antibody portion, of the invention can be combined to include the following: cytokine suppressive anti-inflammatory drug(s) (CSAIDs); antibodies to or antagonists of other human cytokines or growth factors, for example, TNF, LT, IL-1, IL-2, IL-6, IL-7, IL-8, IL-15, IL-16, IL-12, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, or their ligands including CD 154 (gp39 or CD40L).

Some combinations of therapeutic agents may interfere at different points in the autoimmune and subsequent inflammatory cascade; examples include TNF antagonists like chimeric, humanized or human TNF antibodies, D2E7, (U.S. application Ser. No. 08/599,226 filed Feb. 9, 1996, the entire teaching of which is incorporated herein by reference), cA2 (Remicade™), CDP 571, anti-TNF antibody fragments (e.g., CDP870), and soluble p55 or p75 TNF receptors, derivatives thereof, (p75TNFRIgG (Enbrel™) or p55TNFR1gG (Lenercept), soluble IL-13 receptor (sIL-13), and also TNFα converting enzyme (TACE) inhibitors; similarly IL-1 inhibitors (e.g., Interleukin-1-converting enzyme inhibitors, such as Vx740, or IL-1RA, etc.) may be effective for the same reason. Other combinations include Interleukin 11, anti-P7s and p-selectin glycoprotein ligand (PSGL). Yet other combinations involve other key players of the autoimmune response which may act parallel to, dependent on or in concert with IL-12 function. It has been shown that IL-12 and IL-18 have overlapping but distinct functions and a combination of antagonists to both may be most effective. Yet another combination includes non-depleting anti-CD4 inhibitors. Yet other combinations include antagonists of the co-stimulatory pathway CD80 (B7.1) or CD86 (B7.2) including antibodies, soluble receptors or antagonistic ligands.

The antibodies of the invention, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), β-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signalling by proinflammatory cytokines such as TNFα or IL-1 (e.g., IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™) and p55TNFRIgG (Lenercept), sIL-1 RI, sIL-1RII, sIL-6R, soluble IL-13 receptor (sIL-13)) and anti-inflammatory cytokines (e.g., IL-4, IL-10, IL-11, IL-13 and TGFβ). Some combinations include methotrexate or leflunomide and in moderate or severe rheumatoid arthritis cases, cyclosporine. Other agents which may be used in combination with IL-18 antibodies are COX-2 inhibitors. COX-2 inhibitors are known in the art. Specific COX-2 inhibitors are disclosed in WO 01/00229, the entire teaching of which is incorporated herein by reference.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The therapeutically effective amounts of the active protein(s) will be a function of many variables, including the type of anti-IL-18 antibody, the affinity of the antibody for IL-18, any residual cytotoxic activity exhibited by the antibody, the route of administration, the clinical condition of the subject (including the desirability of maintaining a non-toxic level of endogenous IL-18 activity).

A “therapeutically effective amount” is such that when administered, the IL-18 inhibitor results in inhibition of the biological activity of IL-18. The dosage administered, as single or multiple doses, to an individual will vary depending upon a variety of factors, including IL-18 inhibitor pharmacokinetic properties, the route of administration, subject's conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of IL-18 in an individual.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.01-20 mg/kg, or 1-10 mg/kg, or 0.3-1 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### 8. Use of Anti-IL-18 Antibodies

8.1 Uses Generally

Given their ability to bind to IL-18, the anti-IL-18 antibodies, or portions thereof, of the invention can be used to detect IL-18, in one aspect, hIL-18 (e.g., in a sample matrix, in one aspect, a biological sample, such as serum or plasma), using a conventional immunoassay, such as an enzyme linked immunosorbent assays (ELISA), an radioimmunoassay (RIA) or tissue immunohistochemistry. The invention provides a method for detecting IL-18 in a biological sample comprising contacting a sample with an antibody, or antibody portion, of the invention and detecting either the antibody (or antibody portion) bound to IL-18 or unbound antibody (or antibody portion), to thereby detect IL-18 in the sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S, or ³H. Detection of IL-18 in a sample may be useful in a diagnostic context, for example in the diagnosis of a condition associated with increased IL-18, and/or may be useful in identifying a subject who may benefit from treatment with an anti-IL-18 antibody.

Alternative to labeling the antibody, IL-18 can be assayed in a sample by a competition immunoassay utilizing, e.g., rhIL-18 standards labeled with a detectable substance and an unlabeled anti-IL-18 antibody, such as an anti-hIL-18 antibody. In this assay, the sample, the labeled rhIL-18 standards, and the anti-hIL-18 antibody are combined and the amount of labeled rhIL-18 standard bound to the unlabeled antibody is determined. The amount of hIL-18 in the sample is inversely proportional to the amount of labeled rhIL-18 standard bound to the anti-hIL-18 antibody.

The antibodies and antibody portions of the invention are capable of neutralizing IL-18 activity in vitro and in vivo, in one aspect, a hIL-18 activity. Accordingly, the antibodies and antibody portions of the invention can be used to inhibit IL-18 activity, e.g., in a cell culture containing IL-18, in human subjects or in other mammalian subjects having IL-18 with which an antibody of the invention cross-reacts (e.g., primates such as baboon, cynomolgus and rhesus). In a one aspect, the invention provides an isolated human antibody, or antigen-binding portion thereof, that neutralizes the activity of human IL-18, and at least one additional primate IL-18 selected from the group consisting of baboon IL-18, marmoset IL-18, chimpanzee IL-18, cynomolgus IL-18 and rhesus IL-18, but which does not neutralize the activity of the mouse IL-18. In one aspect, the IL-18 is human IL-18. For example, in a cell culture containing, or suspected of containing hIL-18, an antibody or antibody portion of the invention can be added to the culture medium to inhibit hIL-18 activity in the culture.

In another aspect, the invention provides a method for inhibiting IL-18 activity in a subject suffering from a disorder in which IL-18 activity is detrimental. Interleukin 18 plays a critical role in the pathology associated with a variety of diseases involving immune and inflammatory elements.

As used herein, the phrase “a disorder in which IL-18 activity is detrimental” is intended to include diseases and other disorders in which the presence of IL-18 in a subject suffering from the disorder has been shown to be or is suspected of being either responsible for the pathophysiology of the disorder or a factor that contributes to a worsening of the disorder. Accordingly, a disorder in which IL-18 activity is detrimental is a disorder in which inhibition of IL-18 activity is expected to alleviate the symptoms and/or progression of the disorder. Such disorders may be evidenced, e.g., by an increase in the concentration of IL-18 in a biological fluid of a subject suffering from the disorder (e.g., an increase in the concentration of IL-18 in serum, plasma, synovial fluid, etc. of the subject), which can be detected, e.g., using an anti-IL-18 antibody as described above. There are numerous examples of disorders in which IL-18 activity is detrimental. In one aspect, the antibodies or antigen binding portions thereof, can be used in therapy to treat the diseases or disorders described herein. In another aspect, the antibodies or antigen binding portions thereof, can be used for the manufacture of a medicine for treating the diseases or disorders described herein. The use of the antibodies and antibody portions of the invention in the treatment of a few non-limiting specific disorders is discussed further below.

The invention provides pharmaceutical compositions for the treatment of diseases or conditions which require modulation of IL-18 activity. These diseases or conditions include autoimmune diseases, type I diabetes, rheumatoid arthritis, graft rejections, inflammatory bowel disease, sepsis, multiple sclerosis, ischemic heart diseases (including heart attacks), ischemic brain injury, chronic hepatitis, psoriasis, chronic pancreatitis, acute pancreatitis and the like.

Accordingly, anti-IL-18 antibodies or antigen-binding portions thereof, or vectors expressing same in vivo are indicated for the treatment of autoimmune diseases, Type I diabetes, rheumatoid arthritis, graft rejections, inflammatory bowel disease, sepsis, multiple sclerosis, ischemic heart disease including acute heart attacks, ischemic brain injury, chronic hepatitis, psoriasis, chronic pancreatitis and acute pancreatitis and similar diseases, in which there is an aberrant expression of IL-18, leading to an excess of IL-18 or in cases of complications due to exogenously administered IL-18.

8.2 Use in Liver Injury

One aspect of the present invention is to provide for a novel means for treating and/or preventing liver injury. It has been found that an IL-18 inhibitor is effective in the prevention and treatment of liver damages. The invention therefore also relates to the use of an IL-18 inhibitor for the manufacture of a medicament for treatment and/or prevention of liver injury. More specifically, the invention relates to the treatment and/or prevention of liver injuries caused by alcoholic hepatitis, viral hepatitis, immune hepatitis, fulminant hepatitis, liver cirrhosis, and primary biliary cirrhosis.

8.3 Use in Arthritis

It has also been found in accordance with the present invention that an inhibitor of IL-18 is effective in the therapy of arthritis. The therapeutic effect includes decreasing the severity of the disease, as well as preventing the spreading of the disease. The invention therefore relates to the use of an inhibitor of IL-18 for treatment and/or prevention of arthritis. This finding is unexpected, since from the state of the art outlined above, it could not have been concluded that a blockade of one specific factor involved in arthritis, namely interleukin IL-18, would lead to the alleviation of arthritis or even the curing of a diseased arthritic joint.

The term “arthritis” includes all different types of arthritis and arthritic conditions, both acute and chronic arthritis, as defined for example in the Homepage of the Department of Orthopaedics of the University of Washington on Arthritis. Examples for arthritic conditions are ankylosing spondylitis, back pain, carpal deposition syndrome, Ehlers-Danlos-Syndrome, gout, juvenile arthritis, lupus erythematosus, myositis, osteogenesis imperfecta, osteoporosis, polyartheritis, polymyositis, psoriatic arthritis, Reiter's syndrome, scleroderma, arthritis with bowel disease, Behcets's disease, children's arthritis, degenerative joint disease, fibromyalgia, infectious arthritis, Lyme disease, Marfan syndrome, osteoarthritis, osteonecrosis, Pagets Disease, Polymyalgia rheumatica, pseudogout, reflex sympathetic dystrophy, rheumatoid arthritis, rheumatism, Sjogren's syndrome, familial adenomatous polyposis and the like.

Rheumatoid arthritis (RA) causes inflammation in the lining of the joints (the synovial membrane, a one cell layer epithelium) and/or internal organs. The disease tends to persist for many years, typically affects many different joints throughout the body and ultimately can cause damage to cartilage, bone, tendons, and ligaments. The joints that may be affected by RA are the joints located in the neck, shoulders, elbows, hips, wrists, hands, knees, ankles and feet, for example. In many cases, the joints are inflamed in a symmetrical pattern in RA.

RA is prevalent in about 1% of the population in the United States, being distributed within all ethnic groups and ages. It occurs all over the world, and women outnumber men by 3 to 1 among those having RA.

It has been found that the administration of an IL-18 inhibitor significantly diminishes cartilage erosion in a murine model of arthritis. The present invention thus also relates to the use of an inhibitor of IL-18 in the manufacture of a medicament for treatment and/or prevention of cartilage destruction.

### EXAMPLES

### 1. Isolation and Purification of IL-18 Antibodies

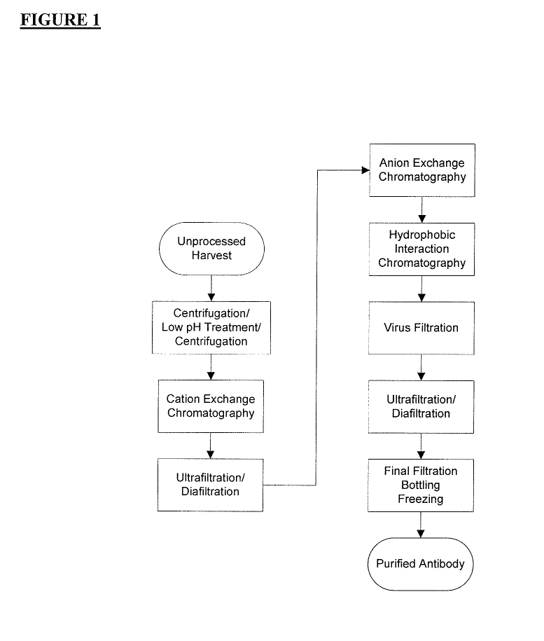

This example provides one scheme of purifying anti-IL-18 antibodies from host cell proteins (HCP) as well as from other impurities. A flow diagram outlining the instant purification process is provided in FIG. 1.

1.1 Primary Recovery with Clarification by Acidification

Primary recovery by centrifugation was used to remove cells and cell debris from a 3000 L production bioreactor harvest. The centrifuge was run at 6900×g at a feed rate of 30 L/min and the clarified supernatant was collected in a pre-sterilized 3000 L harvest tank. The objective of the low pH acidification step is to inactivate adventitious viruses and to prepare the culture supernatant for the subsequent cation capture chromatography step. The centrifuged clarified harvest was adjusted to pH 3.5±0.1 using 3 M citric acid and held at that pH for a period of 1 hr at 20° C. The clarified harvest was then adjusted to pH 4.9±0.1 using 3 M NaOH and held for 16-24 hr at 8° C. The pH-adjusted harvest was brought back to 20° C. and then clarified by centrifugation at 12,750×g at a feed rate of 30 L/min, and the supernatant was collected in a 2000 L tank. Prior to cation exchange chromatography the clarified harvest was passed through a filter train comprising depth filters of nominal pore sizes 0.2-0.8 μm and 0.22 μm hydrophilic filter cartridges. The results for the centrifugation, low pH treatment and re-centrifugation are given in Table 2. The step yield was 69±6% (n=7).

TABLE 2
Centrifugation, Low pH Treatment and Re-centrifugation

| | Lot | | | | | | |
|---|---|---|---|---|---|---|---|
| | BAF04G | BAF05G | BAF06G | BAF07G | BAF08G | BAF01H | BAF02H |
| Antibody at havest (g) | 2225 | 2568 | 2220 | 1929 | 2246 | 2151 | 2153 |
| Antibody in clarified, | 1423 | 1588 | 1575 | 1305 | 1480 | 1712 | 1598 |
| pH treated harvest (g) | | | | | | | |
| Step yield (%) | 64 | 62 | 71 | 68 | 66 | 80 | 74 |

1.2 Cation Exchange Chromatography

The IL-18 antibodies were captured from the clarified harvest by cation exchange chromatograph. In addition, process-related impurities (e.g., host cell proteins, DNA and other process-related impurities) were removed from the process stream. An 80 cm diameter×20 cm long column (bed volume 101 L) was used for this operation. The column was packed with Fractogel™ S resin (EMD Industries, Gibbstown, N.J.) and the asymmetry and Height of an Equivalent Theoretical Plate (HETP) were measured to determine the quality of the packing. Operation of this column was at ambient temperature.

The column was equilibrated using 20 mM Na citrate/citric acid buffer, 65 mM NaCl, pH 5. Depth filtrate was diluted with water to reduce the conductivity to 9±0.5 mS/cm and loaded at a linear velocity of 180 cm/hr. Maximum loading for this chromatography step was set at 27 g protein per liter resin. The column was then washed to baseline with equilibration buffer at a linear velocity of 200 cm/hr. The product was eluted with 20 mM Na citrate/citric acid buffer, 300 mM NaCl, pH 5 at a linear velocity of 125 cm/hr. The column eluate was collected as the absorbance rose above OD 3.0 (A₂₈₀) and continued until the absorbance decreased to an OD 2.0 as the peak tailed. The pooled material was filtered through a 0.8 μm filter followed by a 0.2 μm filter. The results for cation exchange chromatography are given in Table 3. The step yield was 88±6% (n=7) and the purity by SEC HPLC was 98.29±0.52% monomer (n=7).

TABLE 3
Cation Exchange Chromatography

| | Lot | | | | | | |
|---|---|---|---|---|---|---|---|
| | BAP03G | BAP04G | BAP05G | BAP06G | BAP01H | BAP02H | BAP03H |
| | Harvest Lot | | | | | | |
| | BAF04G | BAF05G | BAF06G | BAF07G | BAF08G | BAF01H | BAF02H |
| Fractogel ™ Load Amt. (g) | 1399 | 1507 | 1536 | 1286 | 1434 | 1686 | 1538 |
| Load g protein/L resin | 13.9 | 15.0 | 15.3 | 12.8 | 14.3 | 16.8 | 15.3 |
| Fractogel ™ Eluate Amt. (g) | 1218 | 1398 | 1494 | 1108 | 1193 | 1356 | 1321 |
| Step Yield (g) | 87 | 93 | 97 | 86 | 83 | 80 | 86 |

1.3 Ultratfiltration/Diafiltration

Concentration of the Fractogel™ S eluate was performed using a 30 kD molecular weight cutoff (MWCO) regenerated cellulose acetate ultra filtration membrane cartridge (7 sq. meter total area). Ultrafiltration of the eluate was continued to a final target concentration of 30 g/L. The concentrate was then diafiltered with 6 volumes of 20 mM sodium phosphate buffer, 150 mM NaCl, pH 7.

The UF system was then drained of product and rinsed with diafiltration buffer to recover product held up in the system. The concentrate and wash were combined to produce the diafiltered IL-18 antibodies. Concentrated Fractogel™ SO₃⁻ eluate was immediately 0.2 μm filtered into a holding tank and held at 8° C. until ready to resume processing. The results for the concentration of the Fractogel™ S eluate are given in Table 4. The step was 88±7% (n=7) and the purity by SEC HPLC was 97.67±0.59% monomer (n=7).

TABLE 4
Fractogel S Elutate Concentration

| | Lot | | | | | | |
|---|---|---|---|---|---|---|---|
| | BAP03G | BAP04G | BAP05G | BAP06G | BAP01H | BAP02H | BAP03H |
| Fractogel ™ Eluate Amt. (g) | 1218 | 1398 | 1494 | 1108 | 1193 | 1356 | 1321 |
| Retentate Concentration (g/L) | 24.63 | 21.59 | 16.13 | 20.13 | 21.02 | 21.37 | 19.73 |
| Retentate Amount (g) | 1007 | 1319 | 1131 | 1015 | 1097 | 1231 | 1186 |
| Concentration Yield (%) | 83 | 94 | 76 | 92 | 92 | 91 | 90 |
| SEC HPLC purity (% | 97.61 | 98.27 | 96.97 | 98.25 | 96.77 | 97.97 | 97.84 |
| Monomer) | | | | | | | |

1.4 Anion Exchange Chromatography

Anion exchange chromatography reduces process related impurities such as DNA, viruses, and endotoxins. A 45 cm diameter×30 cm long column (bed volume 48 L) was used this operation. The column was packed with Q Sepharose™ FF resin (GE Healthcare, Piscataway, N.J.) and asymmetry and HETP were measured to determine the quality of the packing. The diluted material was collected in a closed portable stainless steel tank and moved to the Class 10,000 purification suite which was operated at 12° C.

This operation was performed at 12° C. Equilibration of the resin was accomplished with 25 mM trolamine, 40 mM NaCl, pH 8. The maximum protein loading for this chromatography step was set at 60 grams protein per liter of resin. The diluted, filtered, virus inactivated material was designated Q Sepharose™ FF column load. Process-related impurities adsorb to the resin, and antibody flows through the column. The Fractogel™ S eluate concentrate was diluted with two volumes of Q Sepharose™ column load equilibration (50 mM trolamine, pH 8) and loaded onto the column. Loading of the column was performed at 150 cm/hr, and the column flow-through was collected when the A₂₈₀ rose above 0.4 OD. The column was then washed with equilibration buffer and the wash was collected until the A₂₈₀ returned to an OD of 0.6. The flow through and wash were combined to form the eluate product pool. The results for anion exchange chromatography are given in Table 5. The step yield was 92±4% (n=7) and the purity by SEC HPLC was 99.04±0.51% monomer (n=7).

TABLE 5
Anion Exchange Chromatography

| | Lot# | | | | | | |
|---|---|---|---|---|---|---|---|
| | BAP03G | BAP04G | BAP05G | BAP06G | BAP01H | BAP02H | BAP03H |
| Load Amount (g) | 1007 | 1319 | 1131 | 1015 | 1097 | 1231 | 1186 |
| Load g protein/L resin | 21.0 | 27.5 | 23.6 | 21.1 | 22.9 | 25.6 | 24.7 |
| Flow Through and Wash Amt. (g) | 868 | 1249 | 1004 | 957 | 981 | 1175 | 1151 |
| StepYield (%) | 86 | 95 | 89 | 94 | 89 | 95 | 97 |
| SEC HPLC purity (% Monomer) | 99.23 | 99.3 | 97.93 | 99.32 | 99.01 | 99.08 | 99.39 |

1.5 Hydrophobic Interaction Chromatography

Hydrophobic interaction chromatography removes of antibody aggregates and process-related impurities. A 45 cm diameter×15 cm long column (bed volume 24 L) was used for this operation. The column was packed with Phenyl Sepharose™ HP resin (GE Healthcare, Piscataway, N.J.) and asymmetry and HETP were measured to determine the quality of the packing. This unit of operation was also performed at 12° C. in the class 10,000 purification suite.

This operation was performed at 12° C. Equilibration of the resin was accomplished with 20 mM sodium phosphate, 1.1 M ammonium sulfate, pH 7. The maximum protein loading for this step was set at 40 grams protein per liter of resin. The loading of the column was performed at 75 cm/hr. The Q Sepharose™ FTW was diluted with an equal volume of 40 mM sodium phosphate, pH 7, 2.2 M ammonium sulfate, mixed and 0.2 μm filtered. Following loading the column was washed with 20 mM sodium phosphate, pH 7, 1.1 M (NH₄)₂SO₄. The product was eluted by performing a step salt gradient using 9 mM sodium phosphate, pH 7, 0.3 M ammonium sulfate at a linear velocity of 38 cm/hr. Product was collected as the absorbance rose above 1.0 OD at A₂₈₀ and continued until absorbance decreased to 4.0 OD as the peak tailed. One or two cycles were required to process the entire batch of Q Sepharose™ FTW. The results for hydrophobic interaction chromatography are given in Table 6. The step yield was 97±4% (n=7) and the purity by SEC HPLC was 99.30±0.55% monomer (n=7).

TABLE 6
Hydrophobic Interaction Chromatography

| | Lot# | | | | | | |
|---|---|---|---|---|---|---|---|
| | BAP03G | BAP04G | BAP05G | BAP06G | BAP01H | BAP02H | BAP03H |
| Cycle A Load Amount (g) | 922 | 665 | 986 | 933 | 960 | 587 | 575 |
| Cycle B Load Amount (g) | N/A | 652 | N/A | N/A | N/A | 563 | 553 |
| Total Load Amount (A + B) | 922 | 1317 | 986 | 933 | 960 | 1150 | 1128 |
| (g) | | | | | | | |
| Average Load g/L resin | N/A | 27.2 | N/A | N/A | N/A | 23.5 | 23.0 |
| Eluate Amt. (g) | 907 | 1256 | 1034 | 887 | 927 | 1065 | 1101 |
| Step Yield (%) | 98 | 95 | 105 | 95 | 97 | 93 | 98 |
| SEC HPLC purity | 99.51 | 99.57 | 99.05 | 98.20 | 99.31 | 99.69 | 99.80 |
| (% Monomer) | | | | | | | |

1.6 Virus Filtration

Ultipor DV50™ nanofiltration step removes of adventitious viruses ≧50 nm in diameter that may be present in the Phenyl Sepharose™ HP column eluate. This operation was performed at 12° C. Phenyl Sepharose™ HP column eluate was 0.1 μm filtered and passed through a pre-wetted 10″ Ultipor DV50™ filter (Pall Filtron, Northborough, Mass.) at 35 psig. The filter was then flushed with Phenyl Sepharose™ HP column elution buffer to remove any anti-IL-18 retained in the filter housing. The Ultipor DV50™ filtrate was stored in a closed, mobile stainless steel tank at 10-14° C. The results for DV50™ nanofiltration are given in Table 7. The step yield was 96±4% (n=7) and the purity by SEC HPLC was 99.51±0.26% monomer (n=7).

TABLE 7
DV50 Nanofiltration

| | Lot# | | | | | | |
|---|---|---|---|---|---|---|---|
| | BAP03G | BAP04G | BAP05G | BAP06G | BAP01H | BAP02H | BAP03H |
| Feed Amount (g) | 907 | 1256 | 1034 | 887 | 927 | 1065 | 1101 |
| Filtrate Amt. (g) | 842 | 1115 | 1019 | 847 | 950 | 1055 | 1049 |
| Step Yield (%) | 93 | 89 | 98 | 95 | 102 | 99 | 95 |
| SEC HPLC purity | 99.52 | 99.46 | 98.97 | 99.61 | 99.58 | 99.68 | 99.75 |
| (% Monomer) | | | | | | | |

1.7 Final Ultrafiltration/Diafiltration

The UF/DF step is the concentrates of IL-18 antibody, removes ammonium sulfate and diafilters the antibody into formulation buffer. A Millipore 30 kD molecular weight cut-off (MWCO) regenerated cellulose ultrafiltration membrane cartridge (7 sq meters) was used for this step. This step is performed at 12° C. The Ultipor DV50™ nanofiltrate was concentrated to approximately 65 g/L protein. Continuous diafiltration with a minimum of 8 volumes of formulation buffer was then performed. The UF/DF system was then drained of product and rinsed with diafiltration buffer to recover product held up in the system. The concentrate and wash were combined to produce the diafiltered antibody. The antibody sample was then 0.2 μm through a Millipak Opticap™ 10″ filter (0.7 sq meters). The results the ultrafiltration/diafiltration operation are given in Table 8. The step yield was 96±4% (n=7) and the purity by SEC HPLC was 99.51±0.26% monomer (n=7).

TABLE 8
Ultrafiltration/Diafiltration

| | Lot# | | | | | | |
|---|---|---|---|---|---|---|---|
| | BAP03G | BAP04G | BAP05G | BAP06G | BAP01H | BAP02H | BAP03H |
| DV50 ™ Filtrate Amt. (g) | 842 | 1115 | 1019 | 847 | 950 | 1055 | 1049 |
| UF/DFConcentration (g/L) | 65 | 70 | 68 | 75 | 65 | 69 | 70 |
| UF/DF Recovery (g) | 800 | 1110 | 1008 | 858 | 916 | 1034 | 1013 |
| Step Yield (%) | 95 | 100 | 99 | 101 | 96 | 98 | 97 |
| SEC HPLC purity (% | 99.61 | 99.58 | 99.07 | 99.62 | 99.5 | 99.65 | 99.81 |
| Monomer)^{a} | | | | | | | |

^{a}SEC HPLC results based on analysis of drug substance

1.8 Final Filtration, Bottling and Freezing

The formulated antibody was 0.2 μM filtered into 2 L PETG containers and frozen at −80° C. (nominal). The results the ultrafiltration/diafiltration operation are given to Table 9. The step yield was 96±4% (n=7).

TABLE 9
Final Filtration, Bottling and Freezing

| | Lot# | | | | | | |
|---|---|---|---|---|---|---|---|
| | BAP03G | BAP04G | BAP05G | BAP06G | BAP01H | BAP02H | BAP03H |
| DF/DF Amt. (g) | 797 | 1102 | 1004 | 852 | 912 | 1029 | 1009 |
| Bottled Amt. (g) | 764 | 1088 | 937 | 843 | 899 | 992 | 1005 |
| Step Yield (%) | 96 | 99 | 93 | 99 | 99 | 96 | 100 |

### 2. Determination of Host Cell Protein Concentration in Anti-IL-18 Antibody Compositions

This procedure describes the testing methodology for the determination of residual Host Cell Protein concentration in anti-IL-18 antibody samples. Enzyme Linked Immunosorbent Assay (ELISA) is used to sandwich the Host Cell Protein (Antigens) between two layers of specific antibodies. This is followed by the blocking of non-specific sites with Casein. The Host Cell Proteins are then incubated during which time the antigen molecules are captured by the first antibody (Coating Antibody). A second antibody (anti-Host Cell Protein Biotinylated) is then added which fixes to the antigen (Host Cell Proteins). Neutravidin HRP-conjugated is added which binds to the Biotinylated anti-Host Cell Protein. This is followed by the addition of K blue substrate. The chromogenic substrate is hydrolyzed by the bound enzyme conjugated antibody, producing a blue color. Reaction is stopped with 2M H3PO4, changing color to yellow. Color intensity is directly proportional to the amount of antigen bound in the well.

Preparation of 50 mM Sodium Bicarbonate (Coating Buffer), pH 9.4. To a 1 L beaker add: 900 mL Milli-Q water; 4.20 g±0.01 g Sodium Bicarbonate. Stir until completely dissolved. Adjust pH to 9.4 with 1 N NaOH. Transfer to a 1 L volumetric flask and bring to volume with Milli-Q water. Mix by inversion until homogeneous. Filter through a 0.22 μm sterile filter unit. Store at nominal 4° C. for up to 7 days from the date of preparation.

Preparation of 0.104 M Na₂HPO₄×7H₂O, 1.37 M NaCl, 0.027 M KCl, 0.0176 M KH₂PO₄, pH=6.8-6.9 (10×PBS). Add approximately 400 mL of Milli-Q water to a glass beaker. Add 13.94 g±0.01 g of Na₂HPO₄×7H₂O. Add 40.0 g±0.1 g of NaCl. Add 1.00 g±0.01 g of KCl. Add 1.20 g±0.01 g of KH₂PO₄. Stir until homogeneous. Transfer to a 500 mL volumetric flask. QS to 500 mL volume with Milli-Q water. Mix by inversion. Filter through a 0.2 μm sterile filter unit. Store at room temperature for up to 7 days.

Preparation of 1×PBS+0.1% Triton X-100, pH 7.40: (Plate Wash Buffer). In a 4 L graduated cylinder, mix 400 mL 10×PBS (step 5.2) with 3500 mL Milli-Q Water. Check pH, and adjust if necessary to 7.40±0.05 with 1 N HCl or 1 N NaOH. Bring to volume with Milli-Q water. Tightly parafilm the cylinder and mix by inversion until homogeneous. Transfer to a 4 L bottle. Remove 4 mL of the 1×PBS and discard. Add 4 mL of triton X-100 to the 3996 mL of 1×PBS. Place on stir plate and stir to completely dissolve. Filter the amount of plate wash buffer needed for dilution buffer preparation through a 0.22 μm sterile filter unit. Store at room temperature for up to 7 days.

Preparation of Coating Antibody Mixture. Goat anti CHO 599/626/748 (lot # G11201 @1.534 mg/mL), affinity purified. NOTE: Stocks stored at nominal −80° C. in vials. Prepare aliquots. Take out one aliquot per plate at time of use. Immediately before use: Dilute antibody mixture to have a final concentration of 4 μg/mL in cold 50 mM Sodium Bicarbonate as follows. For example: add 31 μLs coating antibody mixture to 11969 μLs cold coating buffer. Mix gently by inversion.

Preparation of Biotinylated goat anti Host Cell Protein Mixture. 599/626/748 (lot# G11202 @0.822 mg/mL): NOTE: Stocks stored at nominal −80° C. in vials. Prepare aliquots. Take out one aliquot per plate at time of use. Immediately before use: Dilute biotinylated antibody mixture to have a final concentration of 1 μg/mL in 37° C.±2° C. Casein as follows. For example: add 14.6 μLs biotinylated antibody mixture to 11985 μLs 37° C.±2° C. Casein. Mix gently by inversion.

Preparation of Neutravidin-HRP. Reconstitute new lots (2 mg/vial) to 1 mg/mL as follows: Add 400 μl of Milli-Q water to the vial, then add 1600 μL 1×PBS, for a total of 2 mL. Vortex gently to mix. Store at nominal −20° C. Prepare aliquots with desired volume so that 1 aliqout per plate is used. Prepare in polypropylene tube. Qualify new lots to determine working concentration. Assign expiry of 6 months from the date of preparation. For example, if the working concentration was determined to be 0.2 μg/mL then prepare as follows. Immediately before use: Thaw an aliquot of Neutravidin-HRP at room temperature. Dilute the 1 mg/mL Neutravidin solution to 0.1 mg/mL (100 μg/mL) with 37° C.±2° C. Casein. For example: Dilute X10, add 50 μL of neutravidin to 450 μL of Casein. Vortex gently to mix. Further dilute the 100 μg/mL solution to 0.2 μg/mL with 37° C.±2° C. Casein. For example: Dilute X500, add 24 μL neutravidin (100 μg/mL) to 11976 μL of Casein. Vortex gently to mix.

Preparation of 5.7 2M Phosphoric Acid (Stop Solution). Prepare a 2 M Phosphoric acid solution from concentrated phosphoric acid as follows. From the % phosphoric acid stated on the label, density (1.685 g/mL) and formula weight (98 g/mole), calculate the volume of concentrated phosphoric acid needed to prepare 500 mL of 2M phosphoric acid. Add the volume of concentrated phosphoric acid calculated above to the flask. Bring to volume with Milli-Q water and mix by inversion until homogeneous. Store at ambient temperature for up to 6 months from the date of preparation.

Preparation of Dilution Buffer (Casein diluted X100 in 1×PBS+0.1% Triton X100, pH 7.4). Dilute 37° C.±2° C. Casein X100 in 0.22 μm sterile filtered 1×PBS+0.1% Triton X100, pH 7.4 (from above). For example: Add 1 mL of 37° C.±2° C. Casein to 99 mL 0.22 μm sterile filtered 1×PBS+0.1% Triton X100, pH 7.4. Mix well. Prepare fresh for each use.

Preparation of Standards. Host cell Protein Standards (Antigen Standards) (lot # G11203 @1.218 mg/mL): NOTE: Stocks stored at nominal −80° C. in 70 μL aliquots. Thaw an aliquot at room temperature. Perform serial dilutions in polypropylene tubes using Dilution buffer.

Preparation of Samples. In polypropylene tubes, dilute final bulk samples to 24 mg/mL in Dilution Buffer. Record concentration. NOTE: Use the solutions below to prepare spiked samples and to prepare the 12 mg/mL solutions referenced below. In polypropylene microtubes, further dilute the 24 mg/mL solutions to 12 mg/mL in Dilution Buffer. Load triplicate wells for each of the 12 mg/mL solutions on the plate for a total of 6 wells.

Preparation of Spike. In a polypropylene microtube, prepare a 10 ng/mL Host Cell Protein spike from the 20 ng/mL standard prepared above by diluting it 2× with Dilution Buffer. Load three wells for the 10 ng/mL spike solution onto the plate. Use the 20 ng/mL standard solution from step 6.1 for spiking samples.

Preparation of Spiked Samples. In polypropylene microtubes, spike 300 μL of each 24 mg/mL final bulk solution with 300 μL of the 20 ng/mL spike solution (6.1). Load triplicate wells for each spiked sample solution for a total of 6 wells.

Preparation of Control. A control range must be set for every new control stock solution, before use in routine testing. Control Stock: Prepare 150 μL aliquots of a batch of ABT-874 Drug Substance Concentrate and store frozen at nominal −80° C. for up to three years.

Preparation of Working Control. Thaw an aliquot of control at room temperature. In polypropylene tubes, dilute control to 24 mg/mL with Dilution Buffer. In polypropylene microtubes, further dilute the 24 mg/mL control solution with dilution buffer to 12 mg/mL. Prepare a single dilution and load control into 3 wells of the plate.

ELISA procedures. Fill plate wash bottle with plate wash buffer (refer to step 5.3, 1×PBS+0.1% Triton X-100). Prime plate washer. Check the following parameters: Parameters should be set to: Plate Type: 1 For each Cycle (a total of 5 cycles): Volume: 400 μls; Soak Time: 10 seconds; Asp. Time: 4 seconds.

Assay Procedure. Coat plates with 100 μL/well of 4 μg/mL goat coating antibody mixture in cold 50 mM Sodium Bicarbonate. Tap the side of the plate until the coating solution covers the bottom of the wells uniformly, cover with sealing tape and incubate at nominal 4° C. while shaking on plate shaker (or equivalent) at speed 3 for 18 hours±1 hour. After overnight incubation, remove plate from refrigerator and allow to equilibrate to room temperature. Shake out coating. Blot plate on paper towels. Block with 300 μL/well of 37° C.±2° C. Casein, cover with sealing tape and incubate at 37° C.±2° C. while shaking on Lab-line Environ plate shaker (or equivalent) at 80 rpm±5 rpm for 1 hour. Prepare standard, sample, control, spike, and spiked samples during blocking incubation. Wash the plate 5 times with Wash Buffer. Blot plate on paper towels. Using an 8-channel pipette, pipet 100 μL/well of standards, samples, spikes, spiked samples, and control into triplicate wells of the plate. Pipette 100 μL/well of Dilution Buffer into all empty wells of the plate to serve as blanks. Cover with sealing tape and incubate at 37° C.±2° C. while shaking on Lab-line Environ plate shaker (or equivalent) at 80 rpm±5 rpm for 1 hour. Fill out a template to use as a guide when loading plate.

Plate Reader Set-Up. Set up template, entering concentrations for standards. Do not enter dilution factors for samples, control, spike, or spiked samples. Assign the wells containing diluent as blanks to be subtracted from all wells. Wash the plate 5 times with Wash Buffer. Blot plate on paper towels. Add 100 μL/well biotinylated goat antibody. Cover with sealing tape and incubate at 37° C.±2° C. while shaking on Lab-line Environ plate shaker (or equivalent) at 80 rpm±5 rpm for 1 hour. Wash the plate 5 times with Wash Buffer. Blot plate on paper towels. Add 1004/well Neutravidin-HRP conjugate solution. Cover with sealing tape and incubate at 37° C.±2° C. while shaking on Lab-line Environ plate shaker (or equivalent) at 80 rpm±5 rpm for 1 hour. Wash the plate 5 times with Wash Buffer. Blot plate on paper towels. Add 100 L/well cold K-Blue substrate, cover with sealing tape and incubate at room temperature for 10 minutes (start timer as soon as substrate is added to first row), while shaking speed 3 on Lab-line titer plate shaker (or equivalent). Stop the reaction by adding 100 μL/well 2M Phosphoric Acid (Step 5.7). Place plate on a plate shaker at speed 3 for 3-5 minutes. Read plate at 450 nm.

Data Analysis and Calculations. NOTE: Only samples, spikes, spiked samples, and control, with optical densities falling within the practical quantitation limit (2.5 ng/mL standard) of the standard curve and meeting the % CV or % difference criteria stated below, are accepted. If sample OD's fall below the 2.5 ng/mL standard, result should be reported as less than 2.5 ng/mL. This value should then be divided by the diluted sample concentration (12 mg/mL) to report value in ng/mg. If sample is high in host cell concentration causing the non-spiked and/or the spiked sample to be above standard curve, report value as >100 ng/mL. This value should then be divided by the diluted sample concentration (12 mg/mL) to report value in ng/mg. Consider sample value zero for spike recovery calculations when the sample is below the 2.5 ng/mL standard.

Standard Curve. Standard concentrations should be entered into the protocol template. A quadratic curve fit is used. Coefficient of determination must be =0.99 and the % CV between triplicate wells must be =20%. If this criteria is not met: One standard (1 level, 3 wells) may be dropped. If the 1.25 ng/mL is dropped, only samples and spiked samples with optical densities falling within the 2.5 ng/mL and 100 ng/mL (the remaining standard curve points) optical densities are acceptable. Additionally, for the triplicates of each standard level, if a single well is clearly contaminated or shows low binding, it may be dropped. If a well is dropped from a standard level, the remaining replicates must have a % difference =20%. The % CV for the lowest standard, which shows OD values close to the background (blanks) of the plate, should be =30%. If one well is dropped, the % difference for the remaining replicates must be =35%. If the lowest standard is dropped, only samples and spiked samples with optical densities falling within the remaining standard curve level optical densities are acceptable.

Samples. % CV should be =20% between triplicate wells. Report % CV between triplicate wells. One well from each sample dilution may be dropped. The remaining replicates must have a % difference of =20%. Note: If non-spiked sample OD is below the 2.5 ng/mL standard OD the % difference criteria does not apply to the non-spiked results. Refer to calculation above. Calculate actual Host Cell Concentration in ng/mg from the mean (ng/mL) value as follows: CHO Host Cell Protein (ng/mg)=Mean “Non-spiked sample result (ng/mL)”_Diluted sample concentration (12 mg/mL).

Spikes. % CV should be =20% between triplicate wells. Record % CV. One well from the spike may be dropped. The remaining points must have a % difference =20%. Refer to calculation in above. Report host cell concentration in ng/mL. This result will be used in spike recovery calculations. The resulting concentration for the spike (ng/mL) must be ±20% of the theoretical spike concentration. Record result and indicate Pass or Fail. If the spike result is not within 20% of theoretical, the assay must be repeated. Mean Spike Concentration (ng/mL)×100=must be 100%±20% 10 ng/mL.

Spiked Samples. % CV should be =20% between triplicate wells. Record % CV between triplicate wells. One well from each spiked sample dilution may be dropped. The remaining replicates must have a % difference of =20%. Refer to calculation above. Report “Spiked sample result” for each dilution in ng/mL. Record % difference between duplicate dilutions. The % difference between dilutions should be =25%. These results will be used in the spike recovery calculations. Calculate % Spike Recovery for each dilution set using the formula below: % Spike Recovery=Spiked sample value−Non-Spiked Sample Value×100 Spike Value. NOTE: (1) If non-spiked sample value OD's fall below the 2.5 ng/mL standard consider value as zero in % spike recovery calculation. % Spike recovery must be 100%±50% (50%−150%) for each dilution for each sample. Record results and Pass/Fail.

Control. % CV should be =20% between triplicate wells. Record % CV result. One well from the control may be dropped. The remaining replicates must have a % difference of =20%. Refer to calculation above. Report Host Cell concentration in the control in ng/mL. Calculate Host Cell concentration in ng/mg as follows: Host Cell Protein (ng/mg)=Control Host Cell Protein result in ng/mL.

Various publications are cited herein, the contents of which are hereby incorporated by reference in their entireties.

What is claimed is:

1. A method for producing a host cell-protein (HCP) reduced IL-18 antibody preparation from a sample mixture comprising an antibody and at least one HCP, said method comprising:
(a) subjecting said sample matrix to a reduction in pH thus forming a primary recovery sample, wherein said reduction in pH is between about 3.0 to about 4.0;
(b) adjusting said primary recovery sample to a pH between about 4.5 to about 5.5 followed by applying said primary recovery sample to an ion exchange resin and collecting an ion exchange sample;
(c) applying said ion exchange sample to a hydrophobic interactive chromatography (HIC) resin and collecting an HIC sample, wherein said HIC sample comprises said HCP-reduced antibody preparation.

2. The method of claim 1, wherein said reduction in pH is accomplished by admixing a suitable acid with said sample mixture, and wherein said suitable acid is selected from the group consisting of citric acid, acetic acid, caprylic acid, and the like.

3. The method of claim 1, wherein said ion exchange resin is either an anion exchange resin or a cation exchange resin.

4. The method of claim 3, wherein said ion exchange resin is a cation exchange resin.

5. The method of claim 4, wherein said cation exchange resin is selected from the group consisting of Fractogel, carboxymethyl (CM), sulfoethyl (SE), sulfopropyl (SP), phosphate (P) and sulfonate (S).

6. The method of claim 5, wherein said cation exchange resin is Fractogel.

7. The method of claim 3, wherein said ion exchange resin is an anion exchange resin.

8. The method of claim 7, wherein said anion exchange resin is selected from the group consisting of Q sepharose, diethylaminoethyl (DEAF), quaternary aminoethyl (QAE), and quaternary amine (Q) groups.

9. The method of claim 8, wherein said anion exchange resin is Q-sepharose.

10. The method of claim 1, wherein said ion exchange step comprises a first ion exchange step and a second ion exchange step.

11. The method of claim 10, wherein said first ion exchange step is a cation exchange step followed by a second anion exchange step.

12. The method of claim 10 further comprising an intermediate step, wherein said intermediate step is a filtration step occurring between said first and said second ion exchange step.

13. The method of claim 12, wherein said filtration step is accomplished by capture ultrafiltration/diafiltration.

14. The method of claim 1, wherein said HIC is accomplished using a column comprising one or more hydrophobic groups.

15. The method of claim 14, wherein said one or more hydrophobic groups are selected from the group consisting of alkyl-, aryl-groups, and a combination thereof.

16. The method of claim 14, wherein said column is selected from the group consisting of phenyl sepharose (such as Phenyl Sepharose™ 6 Fast Flow column, Phenyl Sepharose™ High Performance column), Octyl Sepharose™ High Performance column, Fractogel™ EMD Propyl, Fractogel™ EMD Phenyl columns, Macro-Prep™ Methyl, Macro-Prep™ t-Butyl Supports, WP HI-Propyl (C₃)™ column, and Toyopearl™ ether, phenyl or butyl columns.

17. The method of claim 16, wherein said column comprises phenyl sepharose.

18. The method of claim 1 further comprising a filtration step, wherein said HIC sample is subjected to filtration to remove viral particles and to facilitate buffer exchange.

19. The method of claim 1, wherein said HCP-reduced antibody preparation comprises an anti-IL-18 antibody or an antigen-binding portion thereof.

20. The method of claim 19, wherein said anti-IL-18 antibody or antigen-binding portion thereof is a humanized antibody, a chimeric antibody, or a multivalent antibody.

21. The method of claim 20, wherein said anti-IL-18 antibody or antigen-binding portion thereof is a humanized antibody.

22. The method of claim 20, wherein said anti-IL-18 antibody or antigen-binding portion thereof is an isolated human antibody that dissociates from human IL-18 with a K_{d} of about 1.34×10⁻⁴ M or less and a K_{off} rate constant of about 0.1 s⁻¹ or less both determined by surface Plasmon resonance.

23. The method of claim 19, wherein said anti-IL-18 antibody or antigen-binding portion thereof neutralizes IL-18 both in vivo and in vitro.

24. The method of claim 1, wherein said preparation is substantially free of HCPs.

25. A method for producing a host cell-protein (HCP) reduced antibody preparation from a sample mixture comprising an antibody and at least one HCP, said method comprising:
(a) subjecting said sample matrix to a reduction in pH thus forming a primary recovery sample, wherein said reduction in pH is from about 3.0 to about 4.0;
(b) adjusting said primary recovery sample to a pH from about 4.5 to about 5.5 followed by applying said primary recovery sample to a cation exchange resin and collecting a cation exchange sample;
(c) applying said cation exchange sample to an anion exchange resin and collecting a anion exchange sample; and
(d) applying said anion exchange sample to a hydrophobic interactive chromatography (HIC) resin and collecting an HIC sample, wherein said HIC sample comprises said HCP-reduced antibody preparation.

26. A method for producing a host cell-protein (HCP) reduced antibody preparation from a sample mixture comprising an antibody and at least one HCP, said method comprising:
(a) subjecting said sample matrix to a reduction in pH thus forming a primary recovery sample, wherein said reduction in pH is from about 3.0 to about 4.0;
(b) adjusting said primary recovery sample to a pH from about 4.5 to about 5.5 followed by applying said primary recovery sample to a cation exchange resin and collecting a cation exchange sample;
(c) subjecting said cation exchange sample to filtration and collecting a filtrate.
(d) applying said filtrate from (c) to an anion exchange resin and collecting an anion exchange sample; and
(e) applying said anion exchange sample to a hydrophobic interactive chromatography (HIC) resin and collecting an HIC sample, wherein said HIC sample comprises said HCP-reduced antibody preparation.

27. A pharmaceutical composition comprising an HCP-reduced antibody preparation produced by the method of claim 1 and a pharmaceutically acceptable carrier.

28. The pharmaceutical composition of claim 27, wherein said antibody is an anti-IL-18 antibody or antigen-binding portion thereof.

29. The pharmaceutical composition of claim 27, wherein said composition is substantially free of HCPs.

30. The pharmaceutical composition of claim 27 used to neutralize IL-18 facilitated disorders.

31. The pharmaceutical composition of claim 30, wherein said disorders are selected from the group consisting of autoimmune diseases, type I diabetes, arthritis, rheumatoid arthritis, graft rejections, inflammatory bowel disease, sepsis, multiple sclerosis, ischemic heart diseases (including heart attacks), ischemic brain injury, chronic hepatitis, psoriasis, chronic pancreatitis, acute pancreatitis, alcoholic hepatitis, viral hepatitis, immune hepatitis, fulminant hepatitis, liver cirrhosis, and primary biliary cirrhosis.

32. The pharmaceutical composition of claim 31, wherein said arthritis is selected from the group consisting of ankylosing spondylitis, back pain, carpal deposition syndrome, Ehlers-Danlos-Syndrome, gout, juvenile arthritis, lupus erythematosus, myositis, osteogenesis imperfecta, osteoporosis, polyartheritis, polymyositis, psoriatic arthritis, Reiter's syndrome, scleroderma, arthritis with bowel disease, Behcets's disease, children's arthritis, degenerative joint disease, fibromyalgia, infectious arthritis, Lyme disease, Marfan syndrome, osteoarthritis, osteonecrosis, Pagets Disease, Polymyalgia rheumatica, pseudogout, reflex sympathetic dystrophy, rheumatoid arthritis, rheumatism, Sjogren's syndrome, familial adenomatous polyposis and the like.

33. The pharmaceutical composition of claim 27 further comprising a non-steroidal or steroidal anti-inflammatory drug.

34. The pharmaceutical composition of claim 33 comprising a non-steroidal anti-inflammatory drug.

35. The pharmaceutical composition of claim 34, wherein said non-steroidal anti-inflammatory drug is selected from the group consisting of ibuprofen, corticosteroids, prednisolone, and the like.

36. The pharmaceutical composition of claim 33 comprising a steroidal anti-inflammatory drug.

37. The pharmaceutical composition of claim 27 further comprising one or more other antibodies or antigen-binding portions thereof.

38. The pharmaceutical composition of claim 27 further comprising a pharmaceutical agent.

39. The pharmaceutical composition of claim 38, wherein said pharmaceutical agent is selected from the group consisting of methotrexate, 6-MP, azathioprine sulphasalazine, mesalazine, olsalazine chloroquinine/hydroxychloroquine, pencillamine, aurothiomalate, azathioprine, cochicine, corticosteroids, β-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, phosphodiesterase inhibitors, adenosine agonists, antithrombotic agents, complement inhibitors, adrenergic agents, agents which interfere with signaling by proinflammatory cytokines such as TNFα or IL-1 (e.g., IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme (TACE) inhibitors, T-cell signaling inhibitors such as kinase inhibitors, metalloproteinase inhibitors, sulfasalazine, azathioprine, 6-mercapto-purines, angiotensin converting enzyme inhibitors, soluble cytokine receptors and derivatives thereof (e.g., soluble p55 or p75 TNF receptors and the derivatives p75TNFRIgG (Enbrel™) and p55TNFRIgG (Lenercept), sIL-1 RI, sIL-1RII, sIL-6R, soluble IL-13 receptor (sIL-13)) and anti-inflammatory cytokines (e.g., IL-4, IL-10, IL-11, IL-13 and TGFβ).

40. The methods of claims 1, 25 and 26, wherein said HCP-reduced antibody preparation comprises one or more anti-IL-18 antibodies or antigen-binding portions thereof and are labeled.

41. The methods of claim 40, wherein said label is radioactive.

42. The methods of claim 41, wherein said radioactive label is selected from the group consisting of ¹²⁵I, ¹³¹I, ³⁵S, and ³H.

43. The methods of claim 40, wherein said label is non-radioactive.

44. The methods of claims 1, 25 and 26, wherein said HCP-reduced antibody preparation comprises one or more anti-IL-18 antibodies or antigen-binding portions thereof and are pegylated.
